(19) United States
(12) Patent Application Publication
(10) Pub. No.: US 2016/0045509 A1
(43) Pub. Date: Feb. 18, 2016

(54) Transient Protection of Hematopoietic Stem and Progenitor Cells Against Ionizing Radiation

(71) Applicant: G1 Therapeutics, Inc., Research Triangle Park, NC (US)

(72) Inventors: Jay Copeland Strum, Hillsborough, NC (US); John Emerson Bisi, Apex, NC (US); Patrick Joseph Roberts, Research Triangle Park, NC (US); Francis Xavier Tavares, Durham, NC (US)

(73) Assignee: G1 Therapeutics, Inc., Research Triangle Park, NC (US)

(21) Appl. No.: 14/926,147

(22) Filed: Oct. 29, 2015

Related U.S. Application Data
(63) Continuation of application No. 14/213,382, filed on Mar. 14, 2014, now abandoned.
(60) Provisional application No. 61/800,214, filed on Mar. 15, 2013.

(51) Int. Cl. A61K 31/519; A61K 38/19; A61K 38/18; A61K 45/06

(52) U.S. Cl. CPC A61K 31/519; A61K 45/06; A61K 38/193; A61K 38/1816; A61K 38/196; A61N 2005/1094

(57) ABSTRACT

This invention is in the area of improved compounds and methods for transiently protecting healthy cells, and in particular hematopoietic stem and progenitor cells (HSPC), from the damage associated with ionizing radiation (IR) exposure using selective radioprotectants.

### RELATED APPLICATIONS

This application is a continuation of U.S. application Ser. No. 14/213,382 filed Mar. 14, 2014, which claims the benefit of provisional U.S. Application No. 61/800,214 filed Mar. 15, 2013. The entirety of these applications is hereby incorporated by reference for all purposes.

### GOVERNMENT INTEREST

The U.S. Government has certain rights in this invention arising from support under Grant No. 5R44AI084284 awarded by the National Institutes of Allergy and Infectious Disease.

### FIELD OF THE INVENTION

This invention is in the area of improved compounds and methods for transiently protecting healthy cells, and in particular hematopoietic stem and progenitor cells (HSPC), from the damage associated with ionizing radiation (IR) exposure using selective radioprotectants.

### BACKGROUND

Ionizing radiation (IR) is an important therapeutic modality to treat a range of cancers and other proliferative disorders such as tumors. Radiation therapy uses high energy radiation to shrink tumors and kill the proliferating cells. X-rays, gamma rays, and charged particles are typical kinds of ionizing radiation used for cancer treatments. IR causes extensive DNA damage to exposed cells, including both normal cells and abnormally proliferating cells such as cancer and tumor cells.

Therapeutic radiation is generally applied to a defined area of the subject's body which contains abnormal proliferative tissue, in order to minimize the dose absorbed by the nearby normal tissue. It is difficult, however, to selectively administer therapeutic ionizing radiation to the abnormal tissue. Thus, normal tissue proximate to the abnormal tissue is also exposed to potentially damaging doses of ionizing radiation throughout the course of treatment. There are also some treatments that require exposure of the subject's entire body to the radiation, in a procedure called “total body irradiation” (TBI).

Numerous methods have been designed to reduce normal tissue damage while still delivering effective therapeutic doses of ionizing radiation. These techniques include brachytherapy, fractionated and hyper-fractionated dosing, complicated dosing scheduling and delivery systems, and high voltage therapy with a linear accelerator. Such techniques, however, only attempt to strike a balance between the therapeutic and undesirable effects of the radiation and full efficacy has not been achieved.

In addition, exposure to IR may occur through occupational, environmental, or disaster or terroristic events. For example, occupational doses of ionizing radiation can be received by persons whose job involves exposure to radiation, for example in the nuclear power and nuclear weapons industry. Incidents such as the 1979 accident at Three Mile Island or 2011 accident at the Fukushima nuclear power plant, both of which released radioactive material into the reactor containment building and surrounding environment, illustrate the potential for harmful exposure. Intentional infliction of harmful radiation can occur during war and aggression.

Hematologic toxicity (i.e., IR-induced bone marrow suppression), resulting in myelosuppression, can be a limiting side-effect associated with radiation therapy treatments, resulting in a stoppage, delay, or reduction of treatment until the side-effects subside. Furthermore, hematological toxicity is a major source of morbidity following acute exposure to high doses of radiation. In particular, proliferating hematopoietic stem cells and progenitor cells (HSPCs) within the bone marrow are particularly sensitive to IR, and IR damage to these cells reduces their ability to reconstitute the hematological cell lineages. For example, exposure to high levels of IR such as total body irradiation (TBI) is associated with acute and chronic myelosuppressive hematological toxicities, such as anemia, neutropenia, thrombocytopenia, and lymphcytopenia.

The cytotoxicity of IR, however, is largely cell cycle dependent. In healthy cells, cell division occurs in the context of a highly regulated concert of molecular events known as the cell cycle. The cell cycle is divided into four distinct phases: DNA synthesis (S phase), mitosis (M phase), and the gaps of varying length between these periods called G1 and G2. Non-dividing cells remain in a resting or quiescence stage named G0 before they re-enter into phase G1. Early G1 and late S phases are relatively radioresistant. Conversely, the G1/S transition and G2/M phases are relatively radiosensitive (see Sinclair W K, Morton R A. X-ray sensitivity during cell generation cycle of cultured Chinese hamster cells. Radiat. Res. 1966; 29(3):450-474; Terasuna T, Tolmach L J. X-ray sensitivity and DNA synthesis in synchronous populations of HeLa cells. Science, 1963; 140:490-92.). Transversing from G1 to S phase while harboring DNA damage is particularly toxic. As a result of DNA damage induced by IR, persistent proliferation in the setting of unrepaired DNA damage can be fatal to replicating cells (Little J B. Repair of sub-lethal and potentially lethal radiation damage in plateau phase cultures of human cells. Nature, 1969; 224(5221):804-806.). It has been shown that an extended period of G1 after exposure to DNA-damaging agents enhances resistance to such agents, possibly by allowing for greater DNA repair prior to G1/S transversal (Elkind M M, Sutton H. X-ray damage and recovery in mammalian cells in culture. Nature, 1959; 184: 1293-1295; Elkind M M, Sutton H. Radiation response of mammalian cells grown in culture. 1. Repair of x-ray damage in surviving Chinese hamster cells. Radiat Res. 1960; 13: 556-593). Cell cycle arrest allows cells to properly repair these defects, thus preventing their transmission to the resulting daughter cells. If repair is unsuccessful owing to excessive DNA damage, cells may enter senescence or undergo apoptosis.

Hematopoietic stem cells give rise to progenitor cells which in turn give rise to all the differentiated components of blood as shown in FIG. 1 (e.g., lymphocytes, erythrocytes, platelets, granulocytes, monocytes). HSPCs require the activity of CDK4/6 for proliferation (see Roberts et al. Multiple Roles of Cyclin-Dependent Kinase 4/6 Inhibitors in Cancer Therapy. JNCI 2012; 104(6):476-487). Hematopoietic cells, however, display a gradient dependency on CDK4/6 activity for proliferation during myeloid/erythroid differentiation (see Johnson et al. Mitigation of hematological radiation toxicity in mice through pharmacological quiescence induced by CDK4/6 inhibition. J Clin. Invest. 2010; 120(7): 2528-2536). Accordingly, the least differentiated cells (e.g., hematopoietic stem cells (HSCs), multi-potent progenitors (MPPs), and common myeloid progenitors (CMP)) appear to be the most dependent on CDK4/6 activity for proliferation. More differentiated lineages (e.g., granulocyte-monocyte progenitors (GMPs) and megakaryocyte-erythroid progenitors (MEPs)) are less dependent, and even more differentiated myeloid and erythroid cells proliferate independently of CDK4/6 activity.

A number of CDK 4/6 inhibitors have been identified, including specific pyrido[2,3-d]pyrimidines, 2-anilinopyrimidines, diaryl ureas, benzoyl-2,4-diaminothiazoles, indolo[6,7-a]pyrrolo[3,4-c]carbazoles, and oxindoles (see P. S. Sharma, R. Sharma, R. Tyagi, Curr. Cancer Drug Targets 8 (2008) 53-75). For example, WO 03/062236 identifies a series of 2-(pyridin-2-ylamino-pyrido[2,3]pyrimidin-7-ones for the treatment of Rb positive cancers that show selectivity for CDK4/6, including 6-acetyl-8-cyclopentyl-5-methyl-2-(5-piperazin-1-yl-pyridin-2-ylammino)-8H-pyrido-[2,3-d]-pyrimidin-7-one (PD0332991), which is currently being tested by Pfizer/Onyx in clinical trials as an anti-neoplastic agent against estrogen-positive, HER2-negative breast cancer. The clinical trial studies have reported rates of Grade 3/4 neutropenia and leukopenia with the use of PD0332991, resulting in 71% of patients requiring a dose interruption and 35% requiring a dose reduction; and adverse events leading to 10% of the discontinuations (see Finn, Abstract S1-6, SABCS 2012).

VanderWel et al. describe an iodine-containing pyrido[2,3-d]pyrimidine-7-one (CKIA) as a potent and selective CDK4 inhibitor (see VanderWel et al., J. Med. Chem. 48 (2005) 2371-2387).

WO 99/15500 filed by Glaxo Group Ltd discloses protein kinase and serine/threonine kinase inhibitors.

WO 2010/020675 filed by Novartis AG describes pyrrolopyrimidine compounds as CDK inhibitors.

WO 2011/101409 also filed by Novartis describes pyrrolopyrimidines with CDK 4/6 inhibitory activity.

WO 2005/052147 filed by Novartis and WO 2006/074985 filed by Janssen Pharma disclose additional CDK4 inhibitors.

US 2007/0179118 filed by Barvian et al. teaches the use of CDK4 inhibitors to treat inflammation.

WO 2012/061156 filed by Tavares and assigned to G1 Therapeutics describes CDK inhibitors.

WO 2010/132725 filed by Sharpless and assigned to UNC Chapel Hill, describes the use of CDK inhibitors, for example in combination with growth factors.

Stone, et al., Cancer Research 56, 3199-3202 (Jul. 1, 1996) describes reversible, p16-mediated cell cycle arrest as protection from chemotherapy.

Johnson et al. have shown that pharmacological inhibition of CDK4/6 using the CDK4/6 inhibitors 6-acetyl-8-cyclopentyl-5-methyl-2-(5-piperazin-1-yl-pyridin-2-ylammino)-8H-pyrido-[2,3-d]-pyrimidin-7-one (PD0332991) and 2-bromo-12,13-dihydro-5H-indolo[2,3-a]pyrrolo[3,4]carbazole-5,6-dione (2BrIC) exhibited IR protective characteristics in CDK4/6-dependent cell lines. (Johnson et al. Mitigation of hematological radiation toxicity in mice through pharmacological quiescence induced by CDK4/6 inhibition. J Clin. Invest. 2010; 120(7): 2528-2536). In contrast, these CDK4/6 inhibitors did not G1 arrest the CDK4/6 independent Rb-null melanoma cell line A2058, and failed to protect this cell line from IR exposure. Additional experiments indicated that the protective effects to genotoxins using the tested CDK4/6 inhibitors occurred only when the inhibition resulted in G1 arrest, and cells that were in G2 have enhanced sensitivity to DNA damage. Johnson et al. further described the ability of the selective CDK4/6 inhibitors BrIC and PD0332991 to protect HSPCs and improve survival of mice exposed to peri-lethal and lethal TBI compared to untreated controls, including when PD0332991 was administered post-IR exposure as a mitigant.

U.S. Patent Publication 2011/0224221 to Sharpless et al. describes CDK4/6-dependent HSPC protection against IR using PD0332991 and 2BrIC.

Accordingly, it is an object of the present invention to provide improved compounds and methods to protect healthy cells, and in particular hematopoietic stem and progenitor cells, during IR exposure.

### SUMMARY OF THE INVENTION

In one embodiment, improved methods are provided to minimize the effects of ionizing radiation (IR) on hematopoietic stem cells and/or hematopoietic progenitor cells (together referred to as HSPCs) in subjects, typically humans, that will be, are being, or have been exposed to IR.

Specifically, the invention includes administering an effective amount of a compound of Formula I, II, III, IV, or V, or a pharmaceutically acceptable composition, salt, or prodrug thereof, to provide transient G1-arrest of HSPCs in a subject during or following the subject's exposure to IR.

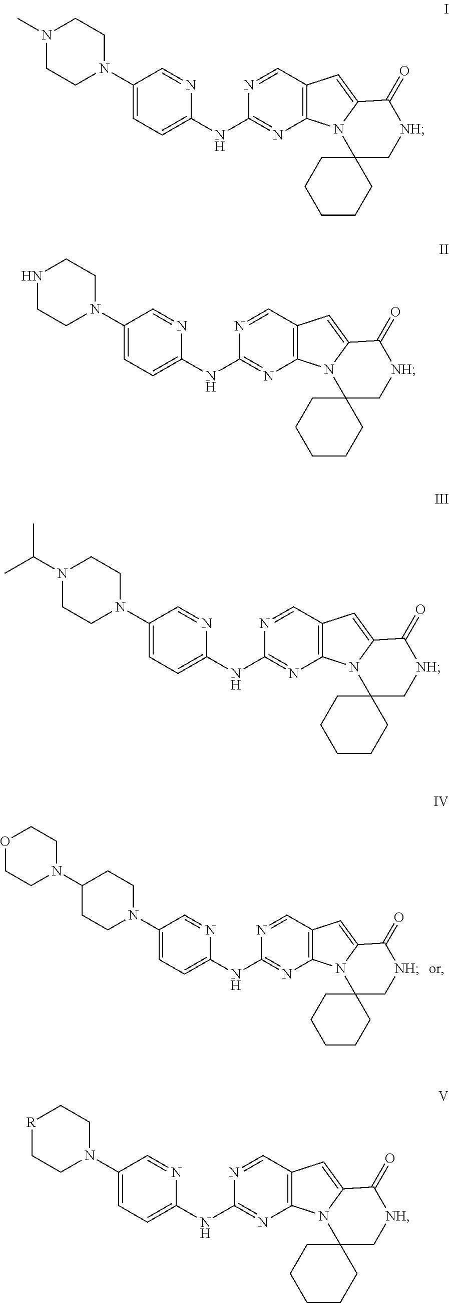

wherein R is C(H)X, NX, C(H)Y, or C(X)₂,

where X is straight, branched or cyclic C₁ to C₅ alkyl group, including methyl, ethyl, propyl, cyclopropyl, isopropyl, butyl, sec-butyl, tert-butyl, isobutyl, cyclobutyl, pentyl, isopentyl, neopentyl, tert-pentyl, sec-pentyl, and cyclopentyl; and

Y is NR₁R₂ wherein R₁ and R₂ are independently X, or wherein R₁ and R₂ are alkyl groups that together form a bridge that includes one or two heteroatoms (N, O, or S); And wherein two X groups can together form an alkyl bridge or a bridge that includes one or two heteroatoms (N, S, or O) to form a spiro compound.

The IUPAC name for Formula I is 2′-((5-(4-methylpiperazin-1-yl)pyridin-2-yl)amino)-7′,8′-dihydro-6′H-spiro[cyclohexane-1,9′-pyrazino[1′,2′:1,5]pyrrolo[2,3-d]pyrimidin]-6′-one; for Formula II is 2′4(5-(piperazin-1-yl)pyridin-2-yl)amino)-7′,8′-dihydro-6′H-spiro[cyclohexane-1,9′-pyrazino[1′,2′:1,5]pyrrolo[2,3-d]pyrimidin]-6′-one; for Formula III is 2′-((5-(4-isopropylpiperazin-1-yl)pyridin-2-yl)amino)-7′,8′-dihydro-6′H-spiro[cyclohexane-1,9′-pyrazino[1′,2′:1,5]pyrrolo[2,3-d]pyrimidin]-6′-one; and for Formula IV is 2′-((5-(4-morpholinopiperidin-1-yl)pyridin-2-yl)amino)-7′,8′-dihydro-6′H-spiro[cyclohexane-1,9′-pyrazino[1′,2′:1,5]pyrrolo[2,3-d]pyrimidin]-6′-one.

The present invention can be used to protect healthy cells during ionizing radiation therapy or radiotherapy for the treatment of any malignant or non-malignant tumor or abnormal cell proliferation, for example, in a solid tumor, including a cancer of the brain, breast, cervix, larynx, lung, pancreas, prostate, skin, spine, stomach, uterus, soft tissue sarcoma, leukemia or lymphoma. The invention can also be used in conjunction with radiotherapy used as a palliative treatment in the absence of a cure for local control of the tumor or symptomatic release, or as a therapeutic treatment to extend the life span of the patient, or total body irradiation performed prior to bone marrow transplant. The invention can also be used to protect healthy cells in connection with radiotherapy for the treatment of non-malignant conditions, such as trigeminal neuralgia, thyroid eye disease, pterygium, or prevention of keloid scar growth or heterotopic ossification. Hyperthermia, or deep tissue heating, is often used in conjunction with radiation to increase the responsiveness of large or advanced tumors to the treatment.

The present invention can also be used to protect healthy cells during ionizing radiation therapy or radiotherapy for the treatment of proliferative disorders, including but not limited to rheumatoid arthritis, lupus, scleroderma, ankylosing spondylitis, asthma, bronchitis and psoriasis. Radiation therapy is also used to treat early stage Dupuytren's disease and Ledderhose disease.

The present invention can further be used to protect people at imminent risk of environmental, occupational or aggression-based radiation exposure or who have recently been exposed to harmful radiation.

The described compounds in a preferred embodiment provide improved protection of CDK-replication dependent HSPCs during or after IR exposure due in part because they (1) exhibit a short, transient G1-arresting effect and (ii) display a rapid, synchronous reentry into the cell cycle by the HSPCs following the cessation of IR exposure or mitigation of IR induced DNA damage. The use of such CDK4/6-specific, short, transient G1-arresting compounds as radioprotectants and radiomitigants allows for an accelerated hematological recovery, reduced hematological cytotoxicity risk due to HSPC replication delay, and/or a minimization of IR induced cell death.

Despite reports using the CDK4/6 inhibitors 2BrIC and PD0332991 to demonstrate radioprotection, it was discovered that these inhibitors may not be the most ideal compounds for use in IR protection strategies. For example, the use of 2BrIC in vivo is limited by its restricted bioavailability. And despite the relative selectivity for CDK4/6 exhibited by PD0332991, the compound has a relatively long-acting intra-cellular effect (see Roberts et al. Multiple Roles of Cyclin-Dependent Kinase 4/6 Inhibitors in Cancer Therapy. JCNI 2012; 104(6):476-487 (FIG. 2A)), extending the transiency of HSPC G1 arrest beyond what may be necessary for sufficient protection from IR exposures. Such a long acting effect delays the proliferation of HSPC cell lineages necessary to reconstitute the hematological cell lines that are adversely affected by IR or are cycled out during their natural life-cycle. The long-acting G1 arrest provided by PD0332991 may limit its use as a potential radioprotectant in subjects whose radiotherapeutic treatment regime or IR exposure requires a rapid reentry into the cell cycle by HSPCs in order to reconstitute the erythroid, platelet, and myeloid cells (monocyte and granulocyte) adversely affected by IR or acute HSPC G1-arrest in order to limit myelosuppressive or hematologic toxicity effects. Furthermore, PD0332991 may be limited in its use as a radioprotectant in subjects exposed to IR at regular and repeated intervals, as it may limit the ability of these subjects' HSPCs to reenter the cell-cycle quickly before it would be necessary to arrest them again prior to the subject's next IR exposure cycle.

Therefore, in an alternative embodiment, the invention includes methods of administering compounds and compositions in an effective amount to a host in need thereof which display one or any combination of the following factors which provide an improved therapeutic effect (either alone or in any combination thereof, each of which is considered specifically and independently described): i) wherein a substantial portion of the CDK4/6-replication dependent HSPC cells (e.g. at least 80% or greater) return to or approach pre-treatment baseline cell cycle activity (i.e., reenter the cell-cycle) in less than 24 hours, 30 hours, or 36 hours from the last administration of the CDK4/CDK6 inhibitory drug in humans or for example, using the protocol described in the Example herein; ii) wherein a substantial portion of the HSPCs reenter the cell-cycle synchronously in less than 24 hours, 30 hours, or 36 hours from the last administration of the CDK4/CDK6 inhibitor; (iii) wherein the dissipation of the inhibitor's CDK4/6 inhibitory effect occurs in less than 24 hours, 30 hours, or 36 hours from the administration of the inhibitor; (iv) wherein the CDK4/6 inhibitor has an IC50 for CDK4 and/or CDK6 inhibition that is more than 1500 times less than its IC50 concentration for CDK2 inhibition; (v) wherein a substantial portion of the HSPCs return to or approach pre-treatment baseline cell cycle activity (i.e., reenter the cell-cycle) in less than 24 hours, 30 hours, or 36 hours from the dissipation of the inhibitor's CDK4/6 inhibitory effect; (vi) wherein the pre-treatment baseline cell cycle activity (i.e. reenter the cell-cycle) within less than about 24 hours, about 30 hours, or about 36 hours from the point in which the CDK4/6 inhibitor's concentration level in the subject's blood drops below a therapeutic effective concentration; or (vii) wherein a substantial portion of the HSPCs reenter the cell-cycle synchronously in less than 24 hours, 30 hours, or 36 hours from the last exposure to IR.

In an alternative embodiment, it has been discovered that an optimal drug for radioprotection and radiomitigation is a CDK4/6 inhibitor that is selected which allows HSPC CDK4/6 dependent cells to return to baseline cell cycle in less than 24, 36, or 40 hours under the following conditions: (i) CDK4/6 dependent human fibroblast cells are pretreated with the CDK4/6 inhibitor such that greater than 85% are growth arrested in G0/G1; (ii) the CDK4/6 inhibitor is removed and cells are monitored at 24, 36, 40, and 48 hours post inhibitor removal for return to baseline cell cycle; (iii) and the baseline cell cycle is defined as the proportion of cells in G0/G1 versus S phase as measured by propidium iodide DNA staining of untreated cells compared to treated cells.

CDK4/6 inhibitors useful in the present invention can be administered to the subject prior to exposure to IR, during exposure to IR, after exposure to IR, or a combination thereof. The inhibitors described herein are typically administered in a manner that allows the drug facile access to the blood stream, for example via intravenous injection or sublingual, intraaortal, or other efficient blood-stream accessing route; however, oral or other desired administrative routes can be used. In one embodiment, the compound is administered to the subject less than about 24 hours, 20 hours, 16 hours, 12 hours, 8 hours, or 4 hours or less prior to exposure to IR. In one embodiment, the compound is administered up to 4 hours prior to exposure to IR. Typically, the CDK4/6 inhibitor is administered to the subject prior to exposure to IR such that the compound reaches peak serum levels before or during exposure to IR. In one embodiment, the CDK4/6 inhibitor is administered concomitantly, or closely thereto, with IR exposure. In one embodiment, the CDK4/6 inhibitor can be administered following exposure to IR in order to mitigate HSPC DNA damage associated with IR exposure. If desired, the CDK4/6 inhibitor can be administered multiple times during the IR exposure to maximize inhibition, especially when the IR exposure occurs over a long period. In one embodiment, the CDK4/6 inhibitor is administered up to about 1 hour, up to about 2 hours, up to about 4 hours, up to about 8 hours, up to about 10 hours, up to about 12 hours, up to about 14 hours, up to about 16 hours, up to about 20 hours, up to about 24 hours or greater following IR exposure. In a particular embodiment, the CDK4/6 inhibitor is administered up to between about 12 hours and 20 hours following exposure to IR. In one embodiment, the CDK4/6 inhibitor is administered one or more times following exposure to IR.

The CDK4/6 inhibitors useful in the present invention show a marked selectivity for the inhibition of CDK4 and/or CDK6 in comparison to other CDKs, for example CDK2. CDK4/6 inhibitors useful in the present invention provide for a dose-dependent G1-arresting effect on a subject's HSPCs sufficient to afford radioprotection to targeted HSPCs during IR exposure, while allowing for the synchronous and rapid reentry into the cell-cycle by the HSPCs shortly after IR exposure and/or CDK4/6 inhibitor administration due to the time-limited CDK4/6 inhibitory effect provided by the compounds described herein compared to, for example, PD0332991. Likewise, CDK4/6 inhibitors useful in the present invention provide a dose-dependent mitigating effect on HSPCs that have been exposed to IR, allowing for repair of DNA damage associated with IR exposure and synchronous, rapid reentry into the cell-cycle following dissipation of the CDK4/6 inhibitory effect compared to, for example, PD0332991. In one embodiment, the use of a CDK4/6 inhibitor described herein results in the G1-arresting effect on the subject's HSPCs dissipation following administration so that the subject's HSPCs return to or approach their pre-administration baseline cell-cycle activity within less than about 24 hours, 30 hours, 36 hours, or 40 hours of administration. In one embodiment, the G1-arresting effect dissipates such that the subject's HSPCs return to their pre-administration baseline cell-cycle activity within less than about 24 hours, 30 hours, 36 hours, or 40 hours of administration.

In one embodiment, the use of a CDK4/6 inhibitor described herein results in the G1-arresting effect dissipation such that the subject's HSPCs return to or approach their pre-administration baseline cell-cycle activity within less than 24 hours, 30 hours, 36 hours, or 40 hours of IR exposure. In one embodiment, the G1-arresting effect dissipates such that the subject's HSPCs return to their pre-administration baseline cell-cycle activity within about 24 hours, 30 hours 36 hours, or 40 hours of IR exposure.

In one embodiment, the use of a CDK4/6 inhibitor described herein results in the G1-arresting effect dissipation so that the subject's HSPCs return to or approach their pre-administration baseline cell-cycle activity within less than about 24 hours, 30 hours, 36 hours, or 40 hours from the point in which the CDK4/6 inhibitor's concentration level in the subject's blood drops below a therapeutic effective concentration. In one embodiment, the G1-arresting effect dissipates such that the subject's HSPCs return to their pre-administration baseline cell-cycle activity within less than about 24 hours, 30 hours, 36 hours, 40 hours from the point in which the CDK4/6 inhibitor's concentration level in the subject's blood drops below a therapeutic effective concentration.

CDK4/6 inhibitors useful in the described methods are synchronous in their off-effect, that is, upon dissipation of the G1 arresting effect, HSPCs exposed to a CDK4/6 inhibitor described herein reenter the cell-cycle in a similarly timed fashion. CDK4/6-replication dependent HSPCs that reenter the cell-cycle do so in such a manner that the normal proportion of cells in G1 and S are reestablished quickly and efficiently, within less than about 24 hours, 30 hours, 36 hours, or 40 hours from the point in which the CDK4/6 inhibitor's concentration level in the subject's blood drops below a therapeutic effective concentration. This advantageously allows for a larger number of HSPCs to begin replicating upon dissipation of the G1 arrest compared with asynchronous CDK4/6 inhibitors such as PD0332991.

In addition, synchronous cell-cycle reentry following G1 arrest using a CDK4/6 inhibitors described herein provides for the ability to time the administration of hematopoietic growth factors to assist in the reconstitution of hematopoietic cell lines to maximize the growth factor effect without forcing hematological cells into replication before DNA damage is repaired. As such, in one embodiment, the use of the compounds described herein is combined with the use of hematopoietic growth factors including, but not limited to, granulocyte colony stimulating factor (G-CSF), granulocyte-macrophage colony stimulating factor (GM-CSF), thrombopoietin, interleukin (IL)-12, steel factor, and erythropoietin (EPO), and their derivatives. In one embodiment, the CDK4/6 inhibitor is administered prior to administration of the hematopoietic growth factor. In one embodiment, the hematopoietic growth factor administration is timed so that the CDK4/6 inhibitor's effect on HSPCs has dissipated.

In one aspect, the use of a CDK4/6-inhibitor described herein allows for a HSPC radio-protective regimen for use during standard radio-therapeutic dosing schedules or regimens common in many anti-cancer treatments. For example, the CDK4/6-inhibitor can be administered so that HSPCs are G1 arrested during IR exposure, wherein, due to the rapid dissipation of the G1-arresting effect of the compounds, a significant number of HSPCs reenter the cell-cycle and are capable of replicating shortly after IR exposure, for example, within about 24-48 hours or less, and continue to replicate until administration of the CDK4/6-inhibitor in anticipation of the next IR exposure. In one embodiment, the CDK4/6-inhibitor is administered to allow for the cycling of the HSPCs between G1-arrest and reentry into the cell-cycle to accommodate a repeated-dosing IR treatment regimen, for example, including but not limited to, a 5-times a week IR treatment regimen, a 4 times a week IR treatment regimen, a 3 times a week IR treatment regimen, a 2 times a week IR treatment regimen, or a 1 time a week or less IR treatment regimen, wherein the HSPCs are G1 arrested during IR exposure and a significant portion of the HSPCs reenter the cell-cycle in between IR exposures. In one embodiment, the CDK4/6-inhibitor can be administered in a manner that the subject's HSPCs are G1-arrested during daily IR exposure, for example a 5 times a week IR regimen, but a significant portion of HSPCs reenter the cell-cycle and replicate in between daily treatment. In one embodiment, the CDK4/6-inhibitors can be administered so that the subject's HSPCs are G1-arrested during IR exposure, for example, including but not limited to, a 3, 4, or 5 times a week IR regimen, but a significant portion of HSPCs reenter the cell-cycle and replicate during the off-day periods, for example, over the weekend between a 5 times a week IR exposure regimen. In one embodiment, the CDK4/6 inhibitor is administered such that a subject's HSPC G1-arrest is provided during a daily IR treatment regimen, for example, a 5-times/week IR treatment regimen, a 4-times/week IR treatment regimen, a 3-times/week IR treatment regimen, a 2-times/week IR treatment regimen, or a 1-time/week IR treatment regimen, and the HSPCs are capable of reentering the cell-cycle shortly after IR exposure, for example within 24-48 hours or less of IR exposure, and before administration of the CDK4/6 inhibition in anticipation of the next IR exposure.

In some embodiments, the subject is undergoing therapeutic IR for the treatment of a proliferative disorder or disease such as cancer. In one embodiment, the cancer is a CDK4/6-replication independent cancer. In some embodiments, the cancer is characterized by one or more of the group consisting of increased activity of cyclin-dependent kinase 1 (CDK1), increased activity of cyclin-dependent kinase 2 (CDK2), loss, deficiency, or absence of retinoblastoma tumor suppressor protein (Rb)(Rb-null), high levels of MYC expression, increased cyclin E, and increased cyclin A. In one embodiment, the subject is undergoing therapeutic IR for the treatment of an Rb-null or Rb-deficient cancer, including but not limited to, small cell lung cancer, triple-negative breast cancer, HPV-positive head and neck cancer, retinoblastoma, Rb-negative bladder cancer, Rb negative prostate cancer, osteosarcoma or cervical cancer. In some cases, administration of the inhibitor compound allows for a higher dose of ionizing radiation to be used to treat the disease than the standard dose that would be safely used in the absence of administration of the CDK4/6 inhibitor compound.

In some embodiments, the subject is at risk of being exposed to IR due to an environmental, occupational or aggression-based situation, such as radiological agent exposure during warfare, a radiological terrorist attack, an industrial accident, other occupational exposure, or space travel.

In some embodiments, the subject has already been exposed to IR, for example, including but not limited to, through an environmental or occupational situation, such as radiological agent exposure during warfare, a radiological terrorist attack, an industrial accident, other occupational exposure, or space travel, and the CDK4/6 inhibitors described herein are administered for the purpose of mitigating DNA damage in HSPCs.

In some embodiments, the protected HSPCs include hematopoietic stem cells, including long term hematopoietic stem cells (LT-HSCs) and short term hematopoietic stem cells (ST-HSCs), and hematopoietic progenitor cells, including multipotent progenitors (MPPs), common myeloid progenitors (CMPs), common lymphoid progenitors (CLPs), granulocyte-monocyte progenitors (GMPs) and megakaryocyte-erythroid progenitors (MEPs). In some embodiments, administration of the inhibitor compound provides temporary, transient pharmacologic quiescence of hematopoietic stem and/or hematopoietic progenitor cells in the subject.

The methods described herein using a CDK4/6 inhibitor are also capable of reducing long-term hematologic toxicity, that is, the use of the CDK4/6 inhibitors described herein prior to, during, or after IR exposure reduces the occurrence or development of long-term hematological toxicities associated with IR exposure. In some embodiments, the reduction in long-term hematological toxicity is associated with the ability of HSPCs that are G1-arrested during IR exposure to rapidly and synchronously renter the cell-cycle shortly after cessation of IR exposure and replicate, including replicating between successive or repeated IR exposures.

Administration of a CDK4/6 inhibitor as described herein can result in reduced anemia, reduced lymphopenia, reduced thrombocytopenia, or reduced neutropenia compared to that typically expected after, common after, or associated with exposure to ionizing radiation in the absence of administration of the CDK4/6 inhibitor. The use of the CDK4/6 inhibitors as described herein may result in a faster recovery from bone marrow suppression associated with long-term use of CDK4/6 inhibitors, including but not limited to, myelosuppression, anemia, lymphopenia, thrombocytopenia, or neutropenia, following the cessation of use of the CDK4/6 inhibitor. In some embodiments, the use of a CDK4/6 inhibitor as described herein results in reduced or limited bone marrow suppression associated with long-term use of CDK4/6 inhibitors, such as myelosuppression, anemia, lymphopenia, thrombocytopenia, or neutropenia.

In aspects of the invention, the CDK4/6 inhibitor used in the aspects of the invention described herein is the compound of Formula I, II, III, IV, or V. In some embodiments, the subject or host is a mammal, including a human. The compound can be administered to the subject by any desired route, including intravenous, sublingual, buccal, oral, intraaortal, topical, intranasal or via inhalation.

In an alternative embodiment, a CDK4/6 inhibitory compounds as described in U.S. Provisional Application No. 61/949,786, incorporated by reference herewith, can be utilized in the described methods.

The present invention includes the following features:

A. Described compounds, methods, and compositions for reducing the effect of IR on CDK4/6 replication dependent HSPCs in a subject undergoing treatment for a CDK4/6-replication independent cancer, for example a Rb-null or Rb-deficient cancer, comprising administering an effective amount of a CDK4/6 inhibitor prior to treatment with IR, are those wherein a substantial portion of the cells return to or approach pre-treatment baseline cell cycle activity (i.e., reenter the cell-cycle) within less than about 24 hours, 30 hours, 36 hours, or about 40 hours from the last administration of the CDK4/6 inhibitor and wherein the CDK4/6 inhibitor has an IC50 concentration for CDK4 inhibition that is more than about 1500 times less than its IC50 concentration for CDK2 inhibition;

B. Described compounds, methods, and composition are provided for reducing the effect of an IR exposure on CDK4/6 replication dependent HSPCs in a subject undergoing treatment for a CDK4/6-replication independent cancer, for example a Rb-null or Rb-deficient cancer, comprising administering an effective amount of a CDK4/6 inhibitor prior to the administration of IR, wherein a substantial portion of the CDK-replication dependent HSPCs synchronously reenter the cell-cycle within less than about 24 hours, 30 hours, 36 hours, or about 40 hours, following the dissipation of the inhibitor's CDK4/6 inhibitory effect, wherein the CDK4/6 inhibitor has an IC50 concentration for CDK4 inhibition that is more than 1500 times less than its IC50 concentration for CDK2 inhibition;

C. Described compounds, methods, and compositions are provided for reducing the effect of IR exposure on CDK4/6 replication dependent HSPCs in a subject who will be exposed, is being exposed, or has been exposed to IR, the method comprising administering an effective amount of a CDK4/6 inhibitor selected from the group consisting of a compound or composition comprising Formula I, Formula II, Formula III, Formula IV, or Formula V described above. In certain embodiments, the subject's HSPCs return to or approach pre-treatment baseline cell cycle activity (i.e., reenter the cell-cycle) within less than about 24 hours, 30 hours, 36 hours, or 40 hours, from the last administration of the CDK4/6 inhibitor. In certain embodiments, the subject's HSPCs return to or approach pre-treatment baseline cell cycle activity (i.e. reenter the cell-cycle) within less than about 24 hours, about 30 hours, about 36 hours, or about 40 hours, from the dissipation of the CDK4/6 inhibitory effect. The subject's HSPCs return to or approach pre-treatment baseline cell cycle activity (i.e. reenter the cell-cycle) within less than about 24 hours, about 30 hours, about 36 hours, or about 40 hours from the point in which the CDK4/6 inhibitor's concentration level in the subject's blood drops below a therapeutic effective concentration;

D. Pyrazinopyrrolopyrimidine compounds of Formula I, II, III, IV, and V as described herein, or pharmaceutically acceptable compositions, salts, isotopic analogs, or prodrugs thereof, for use in the radioprotection of HSPCs during an IR exposure;

E. Pyrazinopyrrolopyrimidine compounds of Formula I, II, III, IV, and V as described herein, and pharmaceutically acceptable compositions, salts, isotopic analogs, or prodrugs thereof, for use in the radioprotection of HSPCs during an IR therapeutic regimen for the treatment of a proliferative disorder;

F. Pyrazinopyrrolopyrimidine compounds of Formula I, II, III, IV, and V as described herein, or pharmaceutically acceptable compositions, salts, isotopic analogs, or prodrugs thereof, for use in the radioprotection of HSPCs during an IR therapeutic regimen for the treatment of cancer;

G. Pyrazinopyrrolopyrimidine compounds of Formula I, II, III, IV, and V as described herein, or pharmaceutically acceptable compositions, salts, isotopic analogs, or prodrugs thereof, for use in the radioprotection of HSPCs during an IR therapeutic regimen for the treatment of a CDK4/6-replication independent cancer;

H. Pyrazinopyrrolopyrimidine compounds of Formula I, II, III, IV, and V as described herein, or pharmaceutically acceptable compositions, salts, isotopic analogs, or prodrugs thereof, for use in the radioprotection of HSPCs during an IR therapeutic regimen for the treatment of an Rb-null or Rb-deficient cancer;

I. Pyrazinopyrrolopyrimidine compounds of Formula I, II, III, IV, and V as described herein, or pharmaceutically acceptable compositions, salts, isotopic analogs, or prodrugs thereof, for use in the radioprotection of HSPCs during IR exposure associated with an environmental or occupational condition;

J. Pyrazinopyrrolopyrimidine compounds of Formula I, II, III, IV, and V as described herein, and pharmaceutically acceptable compositions, salts, isotopic analogs, and prodrugs thereof, for use in the forced cycling of HSPCs between G1-arrest and replication in coordination with a standard IR therapeutic regimen for a proliferative disorder;

K. Pyrazinopyrrolopyrimidine compounds of Formula I, II, III, IV, and V as described herein, or pharmaceutically acceptable compositions, salts, isotopic analogs, or prodrugs thereof, for use in the forced cycling of HSPCs between G1-arrest and replication in coordination with repeated IR exposures;

L. Pyrazinopyrrolopyrimidine compounds of Formula I, II, III, IV, and V as described herein, or pharmaceutically acceptable compositions, salts, isotopic analogs, or prodrugs thereof, for use in the mitigation of DNA damage to HSPCs following IR exposure;

M. Pyrazinopyrrolopyrimidine compounds of Formula I, II, III, IV, and V as described herein, or pharmaceutically acceptable compositions, salts, isotopic analogs, or prodrugs thereof, for use in combination with hematopoietic growth factors in a subject that will be, is being, or has been exposed to IR;

N. Use of pyrazinopyrrolopyrimidine compounds of Formula I, II, III, IV, and V as described herein, or pharmaceutically acceptable compositions, salts, isotopic analogs, or prodrugs thereof, in the manufacture of a medicament for use in the radioprotection of HSPCs;

O. Use of pyrazinopyrrolopyrimidine compounds of Formula I, II, III, IV, and V as described herein, or pharmaceutically acceptable compositions, salts, isotopic analogs, or prodrugs thereof, in the manufacture of a medicament for use in the mitigation of DNA damage of HSPCs that have been exposed to IR;

P. A pharmaceutical formulation comprising an effective subject-treating amount of pyrazinopyrrolopyrimidine compounds of Formula I, II, III, IV, and V as described herein for the protection against ionizing radiation, or pharmaceutically acceptable compositions, salts, isotopic analog, or prodrugs thereof;

Q. A method for manufacturing a medicament of Formula I, II, III, IV, and V intended for therapeutic use in the radioprotection of HSPCs; and,

R. A method for manufacturing a medicament of Formula I, II, III, IV, and V intended for therapeutic use in the mitigation of DNA damage of HSPCs that have been exposed to IR; and,

S. The compound or composition comprising Formula IV as described herein, or a pharmaceutically acceptable composition, salt, isotopic analog or prodrug thereof.

### BRIEF DESCRIPTION OF THE DRAWINGS

FIG. 1 is a schematic drawing of hematopoiesis showing the hierarchical proliferation of healthy hematopoietic stem cells (HSC) and healthy hematopoietic progenitor cells with increasing differentiation upon proliferation.

FIG. 2A is a graph of the percentage of cells in G2-M phase (open circles), S phase (triangles), G0-G1 phase (squares), <2N (diamonds) vs. variable concentration (nM) of Formula I in tHS68 cells. The CDK4/6-dependent cell line (tHS68) was treated with the indicated concentrations of Formula I for 24 hours. Following treatment of Formula I, cells were harvested and analyzed for cell cycle distribution. As described in Example 3, tHS68 cells show a clean G1 arrest accompanied by a corresponding decrease in the number of cells in S-phase.

FIG. 2B is a graph of the number of tHS68 cells (CDK4/6-dependent cell line) vs. the DNA content of the cells (as measured by propidium iodide). Cells were treated with DMSO for 24 hours, harvested, and analyzed for cell cycle distribution.

FIG. 2C is a graph of the number of WM2664 cells (CDK4/6-dependent cell line) vs. the DNA content of the cells (as measured by propidium iodide). Cells were treated with DMSO for 24 hours, harvested, and analyzed for cell cycle distribution.

FIG. 2D is a graph of the number of A2058 cells (CDK4/6-independent cell line) vs. the DNA content of the cells (as measured by propidium iodide). Cells were treated with DMSO for 24 hours, harvested, and analyzed for cell cycle distribution.

FIG. 2E is a graph of the number of tHS68 cells (CDK4/6-dependent cell line) vs. the DNA content of the cells (as measured by propidium iodide) after treatment with Formula I. Cells were treated with Formula I (300 nM) for 24 hours, harvested, and analyzed for cell cycle distribution. As described in Example 3, treatment of tHS68 cells with Formula I causes a loss of the S-phase peak (indicated by arrow).

FIG. 2F is a graph of the number of WM2664 cells (CDK4/6-dependent cell line) vs. the DNA content of the cells (as measured by propidium iodide) after treatment with Formula I. Cells were treated with Formula I (300 nM) for 24 hours, harvested, and analyzed for cell cycle distribution. As described in Example 3, treatment of WM2664 cells with Formula I causes a loss of the S-phase peak (indicated by arrow).

FIG. 2G is a graph of the number of A2058 cells (CDK4/6-independent cell line) vs. the DNA content of the cells (as measured by propidium iodide) after treatment with Formula I. Cells were treated with Formula I (300 nM) for 24 hours, harvested, and analyzed for cell cycle distribution. As described in Example 3, treatment of A2058 cells with Formula I does not cause a loss of the S-phase peak (indicated by arrow).

FIG. 3 is a Western blot showing the phosphorylation levels of Rb at Ser807/811 and Ser780 after treatment with Formula I. CDK4/6-dependent (tHS68 or WM2664) and CDK4/6-independent cell lines (A2058) were treated with Formula I (300 nM) for the indicated times (0, 4, 8, 16, and 24 hours). MAPK levels are shown as a control for protein levels. Following treatment, cells were harvested and analyzed for Rb-phosphorylation by western blot analysis. As described in Example 4, Formula I treatment resulted in reduced Rb-phosphorylation starting 16 hours after treatment in CDK4/6-dependent cell lines (tHS68 and WM2664), but not in the CDK4/6-independent cell line (A2058).

FIG. 4A is a graph of the percentage of cells in S phase in an Rb-positive cell line (WM2664) or in the Rb-negative small cell lung cancer cell lines (H345, H69, H209, SHP-77, NCI417, or H82) after treatment with DMSO (dark bars) or PD0332991 (light bars). Cells were treated with PD0332991 (300 nM) or DMSO control for 24 hours. Cell proliferation was measured by EdU incorporation and flow cytometry. Data represents 100,000 cell events for each cell treatment. As described in Example 5, the RB-null SCLC cell line was resistant to CDK4/6 inhibition, as no change in the percent of cells in S-phase were seen upon treatment with PD0332991.

FIG. 4B is a graph of the percentage of cells in S phase in an Rb-positive cell line (tHS68) or in the Rb-negative small cell lung cancer cell lines (H345, H69, SHP-77, or H82) after treatment with DMSO (dark bars) or Formula III (lighter bars). Cells were treated with Formula III (300 nM or 1000 nM) or DMSO control for 24 hours. Cell proliferation was measured by EdU incorporation and flow cytometry. Data represents 100,000 cell events for each cell treatment. As described in Example 5, the RB-null SCLC cell line was resistant to CDK4/6 inhibition, as no change in the percent of cells in S-phase were seen upon treatment with Formula III.

FIG. 4C is a graph of the percentage of cells in S phase in an Rb-positive cell line (tHS68) or in the Rb-negative small cell lung cancer cell lines (H345, H209, or SHP-77) after treatment with DMSO (dark bars) or Formula I (lighter bars). Cells were treated with Formula I (300 nM or 1000 nM) or DMSO control for 24 hours. Cell proliferation was measured by EdU incorporation and flow cytometry. Data represents 100,000 cell events for each cell treatment. As described in Example 5, the RB-null SCLC cell line was resistant to CDK4/6 inhibition, as no change in the percent of cells in S-phase were seen upon treatment with Formula I.

FIG. 5 is a graph of EdU incorporation vs. time after administration (hours) of PD0332991 to healthy mice HSPCs and healthy myeloid progenitor cells. PD0332991 (150 mg/kg) was administered by oral gavage to assess the temporal effect of transient CDK4/6 inhibition on bone marrow arrest as reported in Roberts et al. Multiple Roles of Cyclin-Dependent Kinase 4/6 Inhibitors in Cancer Therapy. JCNI 2012; 104(6):476-487 (FIG. 2A). As described in Example 7, a single oral dose of PD0332991 results in a sustained reduction in HSPC EdU incorporation (circles; LKS+) and myeloid progenitor cells EdU incorporation (squares; LKS−) for greater than 36 hours.

FIG. 6A is a graph of the ratio of EdU incorporation into HSPCs (compared to untreated control mice) following oral gavage of Formulas I, II, or III at 150 mg/kg at either 12 or 24 hours post administration. FIG. 6B is a graph of the percentage of EdU positive HSPC cells for mice treated with Formula I at either 12 or 24 hours. Mice were dosed with 50 mg/kg (triangles), 100 mg/kg (squares), or 150 (upside down triangles) mg/kg by oral gavage. FIG. 6C is a graph of the percentage of EdU positive HSPC cells for mice treated with Formula I (150 mg/kg by oral gavage) at either 12, 24, 36 and 48 hours. As described in Example 8, Formula I and GG demonstrated a reduction in EdU incorporation at 12 hours, and started to return to normal levels of cell division by 24 hours.

FIG. 7 is a graph of the percentage of EdU positive HSPC cells for mice treated with either PD0332991 (triangles) or Formula I (upside down triangles) v. time after administration (hours) of the compound. Both compounds were administered at 150 mg/kg by oral gavage and the percentage of EdU positive HSPC cells was measured at 12, 24, 36 or 48 hours. As described in Example 9, a single oral dose of PD0332991 results in a sustained reduction of HSPC proliferation for greater than 36 hours. In contrast, a single oral dose of Formula I results in an initial reduction of HSPC proliferation at 12 hours, but proliferation of HSPCs resumes by 24 hours after dosage of Formula I.

FIG. 8A is a graph of the percentage of cells in the G0-G1 phase of the cell cycle vs. time after washout of the compound (hours) in human fibroblast (Rb-positive) cells. FIG. 8B is a graph of the percentage of cells in the S phase of the cell cycle vs. time after washout of the compound (hours) in human fibroblast (Rb-positive) cells. FIG. 8C is a graph of the percentage of cells in the G0-G1 phase of the cell cycle vs. time after washout of the compound (hours) in human renal proximal tubule epithelial (Rb-positive) cells. FIG. 8D is a graph of the percentage of cells in the S phase of the cell cycle vs. time after washout of the compound (hours) in human renal proximal tubule epithelial (Rb-positive) cells. These cellular wash out experiments demonstrated that the inhibitor compounds of the present invention have a short, transient G1-arresting effect in different cell types. The effect on the cell cycle following washing out of the compounds was determined at 24, 36, 40, and 48 hours. As described in Example 10, the results show that cells treated with PD0332991 (circles) took significantly longer to reach baseline levels of cell division (see cells treated only with DMSO (diamonds)), than cells treated with Formula I (squares), Formula II (triangles), Formula III (X), or Formula IV (X with cross).

FIG. 9A is a graph of plasma drug concentration (ng/ml) vs. time after administration (hours) of Formula I.

FIG. 9B is a graph of plasma drug concentration (ng/ml) vs. time after administration (hours) of Formula II.

FIG. 9C is a graph of plasma drug concentration (ng/ml) vs. time after administration (hours) of Formula III.

FIG. 9D is a graph of plasma drug concentration (ng/ml) vs. time after administration (hours) of Formula IV. Compounds were dosed to mice at 30 mg/kg by oral gavage (diamonds) or 10 mg/kg by intravenous injection (squares). Blood samples were taken at 0, 0.25, 0.5, 1.0, 2.0, 4.0, and 8.0 hours post dosing and the plasma concentrations were determined by HPLC.

FIG. 10 provides the half-life (minutes) of Formula I and PD0332991 in human and animal (monkey, dog, rat, and mouse) liver microsomes. As described in Example 12, PD0332991 has a half-life greater than 60 minutes in each of the species tested. Formula I was determined to have a shorter half-life than PD0332991 in each of the species tested.

FIG. 11 is a series of contour plots showing proliferation (as measured by EdU incorporation after 12 hours) vs. cellular DNA content (as measured by DAPI staining) Representative contour plots show proliferation in WBM (whole bone marrow; top) and HSPCs (hematopoietic stem and progenitor cells; LSK; bottom), as measured by EdU incorporation after 12 hours of no treatment, EdU treatment only, or EdU plus Formula I treatment. As described in Example 13, Formula I reduces proliferation of whole bone marrow and hematopoietic stem and/or progenitor cells.

FIG. 12A is a graph of the percentage of EdU-positive cells in whole bone marrow (WBM) and various hematopoietic stem and progenitor cells (Lin−, LSK, HSC, MPP, or CD28+LSK cell lineages) treated with Formula I (open bars) or untreated (solid bars). As described in Example 13, treatment with Formula I inhibits proliferation of WBM and all HSPC lineages tested. *P<0.05, **P<0.01.

FIG. 12B is a graph of the percentage of EdU-positive cells in whole bone marrow (WBM) and various lineage restricted progenitors (MP, GMP, MEP, CMP, or CLP cell lineages) treated with Formula I (open bars) or untreated (solid bars). As described in Example 13, treatment with Formula I inhibits proliferation of WBM and all lineage restricted progenitors tested. *P<0.05, **P<0.01.

FIG. 13A is a graph of the percentage of EdU-positive cells in T cell populations (Total, CD4+, CD8+, DP, DN, DN1, DN2, DN3, or DN4) treated with Formula I (open bars) or untreated (solid bars). As described in Example 14, treatment with Formula I inhibits proliferation of the CD4+, CD8+, DP, DN, DN1, DN2, DN3, or DN4 T cell populations. *P<0.05, **P<0.01.

FIG. 13B is a graph of the percentage of EdU-positive cells in B cell populations (B220+, B220+sIgM+, Pre-pro-B sIgM-, Pro-B, Pre-B) treated with Formula I (open bars) or untreated (solid bars). As described in Example 14, treatment with Formula I inhibits proliferation of the the various B cell populations (B220+, B220+sIgM+, Pre-pro-B sIgM-, Pro-B, and Pre-B). *P<0.05, **P<0.01.

FIG. 13C is a graph of the percentage of EdU-positive cells in myeloid cell populations (Mac1+Gr1+, Ter119+, or CD41+) treated with Formula I (open bars) or untreated (solid bars). As described in Example 14, treatment with Formula I inhibits proliferation of the Mac1+Gr1+, Ter119+, or CD41+ myeloid cell populations. *P<0.05, **P<0.01.

FIG. 14A is a graph of caspase 3/7 activity (relative % compared to the control) in cell lines treated with Formula I (0, 100 nM, 300 nM, or 1 uM) after irradiation with 6 Gy, 8 Gy, or 10 Gy of ionizing radiation. As described in Example 15, Formula I shows a dose-dependent increase in protection of cells from irradiation induced apoptosis at all three irradiation levels tested.

FIG. 14B is a graph of H2AX foci (relative % compared to the control) in cell lines treated with Formula I (0, 100 nM, 300 nM, or 1 uM) after irradiation with 6 Gy, 8 Gy, or 10 Gy of ionizing radiation. As described in Example 15, Formula I shows a dose-dependent increase in protection of cells from irradiation induced DNA damage at all three irradiation levels tested.

FIG. 15A is a Kaplan-Meier analysis of survival after 7.2 Gy of total body irradiation (TBI) in mice treated with Formula I dosed orally at 150 mg/kg 12 hours post TBI as compared to control mice. As described in Example 16, mice treated with Formula I show a significant improvement in survival rates after total body irradiation.

FIG. 15B is a Kaplan-Meier analysis of survival after 7.5 Gy of total body irradiation (TBI) in mice treated with Formula I dosed orally at 150 mg/kg 12 hours post TBI as compared to control mice. As described in Example 16, mice treated with Formula I show a significant improvement in survival rates after total body irradiation.

FIG. 15C is a Kaplan-Meier analysis of survival after 7.5 Gy of total body irradiation (TBI) in mice treated with two doses of Formula I. Mice were dosed orally at 150 mg/kg 12 hours post TBI and dosed again at 150 mg/kg 24 hours post TBI as compared to control mice. As described in Example 16, mice treated with two doses of Formula I show a significant improvement in survival rates after total body irradiation.

### DETAILED DESCRIPTION OF THE INVENTION

Improved compounds, methods, and compositions are provided to minimize the effect of IR toxicity on CDK4/6 replication dependent hematopoietic stem cells and/or hematopoietic progenitor cells (together referred to as HSPCs) in subjects, typically humans, that will be, are being or have been exposed to IR.

### I. Definitions

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety to the extent authorized by law.

The term “selective CDK4/6 inhibitor” and derivatives thereof means a compound that inhibits only CDK4 activity, only CDK6 activity, or both CDK4 and CDK6 activity at an IC₅₀ molar concentration at least about 1500 times or 1800 times or 2000 times less than the IC₅₀ molar concentration necessary to inhibit to the same degree of CDK2 activity in a standard phosphorylation assay.

The term “and/or” when used in describing two items or conditions, e.g., CDK4 and/or CDK6, refers to situations where both items or conditions are present or applicable and to situations wherein only one of the items or conditions is present or applicable. Thus, a CDK4 and/or CDK6 inhibitor can be a compound that inhibits both CDK4 and CDK6, a compound that inhibits only CDK4, or a compound that only inhibits CDK6.

As described herein, hematopoietic stem and progenitor cells include, but are not limited to, long term hematopoietic stem cells (LT-HSCs), short term hematopoietic stem cells (ST-HSCs), multipotent progenitors (MPPs), common myeloid progenitors (CMPs), common lymphoid progenitors (CLPs), granulocyte-monocyte progenitors (GMPs), and megakaryocyte-erythroid progenitors (MEPs).

As used herein the term “ionizing radiation” refers to radiation of sufficient energy that, when absorbed by cells and tissues, can induce formation of reactive oxygen species and DNA damage. Ionizing radiation can include X-rays, gamma rays, and particle bombardment (e.g., neutron beam, electron beam, protons, mesons, and others). IR is used for purposes including, but not limited to, medical testing and treatment, scientific purposes, industrial testing, manufacturing and sterilization, and weapons and weapons development, nuclear energy and can also be found as an environmental or occupational toxin or used as an assault. Radiation is generally measured in units of absorbed dose, such as the rad or gray (Gy), or in units of dose equivalence, such as rem or sievert (Sv).

By “substantial portion” or “significant portion” is meant at least about 80%. In alternative embodiments, the portion may be 85%, 90% or 95% or greater.

By “induces G1-arrest” is meant that the inhibitor compound induces a quiescent state in a substantial portion of a cell population at the G1 phase of the cell cycle.

By “long-term hematological toxicity” is meant hematological toxicity affecting a subject for a period lasting more than one or more weeks, months or years following exposure of IR. Long-term hematological toxicity can result in bone marrow disorders that can cause the ineffective production of blood cells (i.e., myelodysplasia) and/or lymphocytes (i.e., lymphopenia, the reduction in the number of circulating lymphocytes, such as B- and T-cells). Hematological toxicity can be observed, for example, as anemia, reduction in platelet count (i.e., thrombocytopenia) or reduction in white blood cell count (i.e., neutropenia). In some cases, myelodysplasia can result in the development of leukemia. Long-term toxicity related to ionizing radiation can also damage other self-renewing cells in a subject, in addition to hematological cells. Thus, long-term toxicity can also lead to graying and frailty.

As used herein, the term “prodrug” means a compound which when administered to a host in vivo is converted into the parent drug. As used herein, the term “parent drug” means any of the presently described chemical compounds that are useful to treat any of the disorders described herein, or to control or improve the underlying cause or symptoms associated with any physiological or pathological disorder described herein in a host, typically a human. Prodrugs can be used to achieve any desired effect, including to enhance properties of the parent drug or to improve the pharmaceutic or pharmacokinetic properties of the parent. Prodrug strategies exist which provide choices in modulating the conditions for in vivo generation of the parent drug, all of which are deemed included herein. Nonlimiting examples of prodrug strategies include covalent attachment of removable groups, or removable portions of groups, for example, but not limited to acylation, phosphorylation, phosphonylation, phosphoramidate derivatives, amidation, reduction, oxidation, esterification, alkylation, other carboxy derivatives, sulfoxy or sulfone derivatives, carbonylation or anhydride, among others.

Throughout the specification and claims, a given chemical formula or name shall encompass all optical and stereoisomers, as well as racemic mixtures where such isomers and mixtures exist, unless otherwise noted.

A CDK4/6 inhibitor that is “substantially free” of off-target effects is a CDK4/6 inhibitor that can have some minor off-target effects that do not interfere with the inhibitor's ability to provide protection from cytotoxic compounds in CDK4/6-dependent cells. For example, a CDK4/6 inhibitor that is “substantially free” of off-target effects can have some minor inhibitory effects on other CDKs (e.g., IC₅₀s for CDK1 or CDK2 that are >0.5 μM; >1.0 μM, or >5.0 μM), so long as the inhibitor provides selective G1 arrest in CDK4/6-dependent cells.

By “synchronous reentry into the cell cycle” is meant that HSPC cells in G1-arrest due to the effects of a CDK4/6 inhibitor compound reenter the cell-cycle within relatively the same collective timeframe or at relatively the same rate upon dissipation of the compound's effect. Comparatively, by “asynchronous reentry into the cell cycle” is meant that the HSPC cells in G1 arrest due to the effects of a CDK4/6 inhibitor compound reenter the cell-cycle within relatively different collective timeframes or at relatively different rates upon dissipation of the compound's effect, such as induced by PD 0332991.

The subject treated or exposed to IR is typically a human subject, although it is to be understood the methods described herein are effective with respect to other mammals or vertebrate species. The term subject can include animals such as mice, monkeys, dogs, pigs, rabbits, domesticated swine (pigs and hogs), ruminants, equine, poultry, felines, murines, bovines, canines, and the like.

### Isotopic Substitution

The present invention includes compounds and the use of compounds with desired isotopic substitutions of atoms, at amounts above the natural abundance of the isotope, i.e., enriched. Isotopes are atoms having the same atomic number but different mass numbers, i.e., the same number of protons but a different number of neutrons. By way of general example and without limitation, isotopes of hydrogen, for example, deuterium (2H) and tritium (3H) may be used anywhere in described structures. Alternatively or in addition, isotopes of carbon, e.g., 13C and 14C, may be used. A preferred isotopic substitution is deuterium for hydrogen at one or more locations on the molecule to improve the performance of the drug. The deuterium can be bound in a location of bond breakage during metabolism (an α-deuterium kinetic isotope effect) or next to or near the site of bond breakage (a β-deuterium kinetic isotope effect).

Substitution with isotopes such as deuterium can afford certain therapeutic advantages resulting from greater metabolic stability, such as, for example, increased in vivo half-life or reduced dosage requirements. Substitution of deuterium for hydrogen at a site of metabolic break down can reduce the rate of or eliminate the metabolism at that bond. At any position of the compound that a hydrogen atom may be present, the hydrogen atom can be any isotope of hydrogen, including protium (1H), deuterium (2H) and tritium (3H). Thus, reference herein to a compound encompasses all potential isotopic forms unless the context clearly dictates otherwise. The term “isotopically-labeled” analog refers to an analog that is a “deuterated analog”, a “13C-labeled analog,” or a “deuterated/13C-labeled analog.” The term “deuterated analog” means a compound described herein, whereby a H-isotope, i.e., hydrogen/protium (1H), is substituted by a H-isotope, i.e., deuterium (2H). Deuterium substitution can be partial or complete. Partial deuterium substitution means that at least one hydrogen is substituted by at least one deuterium. In certain embodiments, the isotope is 90, 95 or 99% or more enriched in an isotope at any location of interest. In some embodiments it is deuterium that is 90, 95 or 99% enriched at a desired location.

### II. Hematopoietic Stem Cells and Cyclin-Dependent Kinase Inhibitors

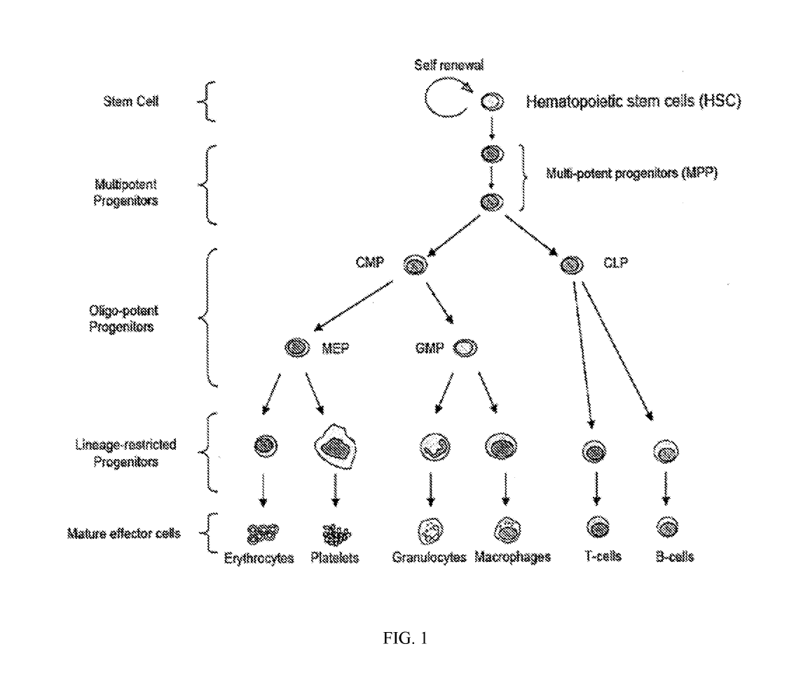

Tissue-specific stem cells are capable of self-renewal, meaning that they are capable of replacing themselves throughout the adult mammalian lifespan through regulated replication. Additionally, stem cells divide asymmetrically to produce “progeny” or “progenitor” cells that in turn produce various components of a given organ. For example, in the hematopoietic system, the hematopoietic stem cells give rise to progenitor cells which in turn give rise to all the differentiated components of blood (e.g., white blood cells, red blood cells, and platelets). See FIG. 1.

Early hematopoietic stem/progenitor cells (HSPC) in the adult mammal require the enzymatic activity of the proliferative kinases cyclin-dependent kinase 4 (CDK4) and/or cyclin-dependent kinase 6 (CDK6) for cellular replication. In contrast, the majority of proliferating cells in adult mammals (e.g., the more differentiated blood-forming cells in the bone marrow) do not require the activity of CDK4 and/or CDK6 (i.e., CDK4/6). These differentiated cells can proliferate in the absence of CDK4/6 activity by using other proliferative kinases, such as cyclin-dependent kinase 2 (CDK2) or cyclin-dependent kinase 1 (CDK1).

The present invention includes methods of protecting healthy cells in a subject, and in particular, hematopoietic cells and/or progenitor cells (HSPCs) from the toxic effects or mitigation of ionizing radiation by the administration of a selective CDK4/6 inhibitor, in particular the described CDK4/6 inhibiting pyrazinopyrrolopyrimidine compounds, having a selective, short, transient G1-arresting effect on HSPCs, the inhibitors providing for sufficient protection of the HSPCs during or after IR exposure to reduce or prevent IR cytotoxicity to the HSPCs and a rapid, synchronous reentry into the cell cycle by the HSPCs following the cessation of IR exposure or mitigation of IR DNA damage. The use of CDK4/6-specific, short, transient G1-arresting effect compounds as radioprotectants and radiomitigants allows for an accelerated hematological recovery and reduced hematological cytotoxicity risk due to HSPC replication delay. In certain embodiments, the CDK4/6 inhibitor administered is selected from the group consisting of a compound or composition comprising Formula I, Formula II, Formula III, Formula IV, Formula V, or a combination thereof.

In certain aspects, compounds, methods, and compositions are provided for reducing or limiting the effect of DNA damaging ionizing radiation on hematopoietic stem and progenitor cells in a subject undergoing treatment for a Rb-null cancer, the method comprising administering an effective amount of a CDK4/6 inhibitor prior to exposure to IR, wherein a substantial portion of the hematopoietic stem and/or progenitor cells return to pre-treatment baseline cell cycle activity (i.e., reenter the cell-cycle) within less than about 24, 30, 36, or 40 hours of administration of the CDK4/6 inhibitor; wherein the CDK4/6 inhibitor has an IC₅₀ CDK4 inhibitory concentration that is at least 1500 times less than its IC₅₀ inhibitory concentration for CDK2. In certain embodiments, the CDK4/6 inhibitor administered is selected from the group consisting of the compound or a composition comprising Formula I, Formula II, Formula III, Formula IV, and Formula V, or a pharmaceutically acceptable composition, salt, isotopic analog, or prodrug thereof.

In certain aspects, compounds, methods, and composition are provided for reducing or limiting the effect of DNA-damaging IR on hematopoietic stem and progenitor cells in a subject undergoing treatment for a RB-null cancer, the method comprising administering an effective amount of a CDK4/6 inhibitor prior to the administration of the IR, wherein a substantial portion of the hematopoietic stem and/or progenitor cells synchronously reenter the cell-cycle within less than about 24, 30, 36, or 40 hours or less following the dissipation of the inhibitor's CDK4/6 inhibitory effect, wherein the CDK4/6 inhibitor has an IC50 CDK4 inhibitory concentration that is at least 1500 times less than its IC₅₀ inhibitory concentration for CDK2. In certain embodiments, the CDK4/6 inhibitor administered is selected from the group consisting of a compound or composition comprising Formula I, Formula II, Formula III, Formula IV, and Formula V, or a pharmaceutically acceptable composition, salt, isotopic analog, or prodrug thereof.

In certain aspects, compounds, methods, and composition are provided for reducing or limiting the effect of DNA-damaging IR on hematopoietic stem and progenitor cells in a subject that has been exposed to IR, the method comprising administering an effective amount of a CDK4/6 inhibitor following exposure to IR, wherein a substantial portion of the hematopoietic stem and/or progenitor cells reenter the cell-cycle synchronously within less than about 24, 30, 36, or 40 hours following the dissipation of the inhibitor's CDK4/6 inhibitory effect, wherein the CDK4/6 inhibitor has an IC₅₀ CDK4 inhibitory concentration that is more than 500 times less than its IC₅₀ inhibitory concentration for CDK2. In certain embodiments, a substantial portion of the hematopoietic stem and/or progenitor cells reenter the cell-cycle synchronously within less than about 24, 30, 36, or 40 hours from the point in which the CDK4/6 inhibitor's concentration level in the subject's blood drops below a therapeutic effective concentration. In certain embodiments, the CDK4/6 inhibitor administered is selected from the group consisting of a compound or composition comprising Formula I, Formula II, Formula III, Formula IV, or Formula V, or a pharmaceutically acceptable composition, salt, isotopic analog, or prodrug thereof.

In certain embodiments, the CDK4/6 inhibitor is a pyrazinopyrrolopyrimidine CDK4/6 inhibitor of Formula I, II, III, IV, or V, or a pharmaceutically acceptable composition, salt, isotopic analog, or prodrug thereof, wherein the protection afforded by the compound is short term and transient in nature, allowing a significant portion of the cells to synchronously renter the cell-cycle quickly following the cessation of IR exposure, for example within less than about 24, 30, 36, or 40 hours. Cells that are quiescent within the G1 phase of the cell cycle are more resistant to the DNA damaging effect of radiation than proliferating cells.

CDK4/6 inhibitory compounds for use in the described methods are highly selective, potent CDK4/6 inhibitors, with minimal CDK2 inhibitory activity. In one embodiment, a CDK4/6 compound for use in the methods described herein has a CDK4/CycD1 IC₅₀ inhibitory concentration value that is >1500 times, >1800 times, >2000 times, >2200 times, >2500 times, >2700 times, >3000 times, >3200 times or greater lower than its respective IC₅₀ concentration value for CDK2/CycE inhibition. In one embodiment, a CDK4/6 inhibitor for use in the methods described herein has an IC₅₀ concentration value for CDK4/CycD1 inhibition that is about <1.50 nM, <1.25 nM, <1.0 nM, <0.90 nM, <0.85 nM, <0.80 nM, <0.75 nM, <0.70 nM, <0.65 nM, <0.60 nM, <0.55 nM, or less. In one embodiment, a CDK4/6 inhibitor for use in the methods described herein has an IC₅₀ concentration value for CDK2/CycE inhibition that is about >1.0 μM, >1.25 μM, >1.50 μM, >1.75 μM, >2.0 μM, >2.25 μM, >2.50 μM, >2.75 μM, >3.0 μM, >3.25 μM, >3.5 μM or greater. In one embodiment, a CDK4/6 inhibitor for use in the methods described herein has an IC₅₀ concentration value for CDK2/CycA IC₅₀ that is >0.80 μM, >0.85 μM, >0.90 μM, >0.95 μM, >0.1.0 μM, >1.25 μM, >1.50 μM, >1.75 μM, >2.0 μM, >2.25 μM, >2.50 μM, >2.75 μM, >3.0 μM or greater. In one embodiment, the CDK4/6 inhibitor for use in the methods described herein are selected from the group consisting of Formula I, Formula II, Formula III, Formula IV, Formula V, or a pharmaceutically acceptable composition, salt, isotopic analog, or prodrug, thereof.

CDK4/6 inhibitors useful in the described methods provide for a short, transient, and reversible G1-arrest of HSPC cells. By having a short-term transient effect, the use of such CDk4/6 inhibitors in a radioprotection or radiomitigation regimen allows for the faster reentry of the HSPCs into the cell cycle following cessation of IR exposure or following mitigation of DNA damage repair compared to, for example, longer acting CDK4/6 inhibitors such as PD0332991. The quicker dissipation of the G1 arresting effect on HSPCs makes such compounds preferable over longer acting CDK4/6 inhibitors in situations where: 1) the subject will be exposed to closely spaced IR treatments, wherein the use of a longer acting CDK4/6 inhibitor would prohibit the cycling of the HSPCs between IR exposures; or 2) IR exposure regimens wherein the long-term G1 arrest of HSPCs is required due to the closely repeated IR exposures, and the subject would benefit from the HSPCs quickly reentering the cell-cycle following cessation of the treatment regime or between breaks in treatment in order to limit HSPC replication delay, thus reducing, limiting, or ameliorating further bone marrow suppression upon cessation of IR exposure. According to the presently disclosed subject matter, radiation protection with the selective CDK4/6 inhibitors described herein can be achieved by a number of different dosing schedules. In addition to multi-dosing schedules or single pretreatment, concomitant treatment can also be effective.

In one embodiment, the CDK4/6 inhibitors described herein are used in HSPC cycling strategies wherein a subject is exposed to regular, repeated IR exposures, wherein HSPCs are G1-arrested when IR exposed and allowed to reenter the cell-cycle before the subject's next IR exposure. Such cycling allows HSPCs to regenerate damaged blood cell lineages in between regular, repeated IR exposures, for example those associated with standard IR treatments for cancer, and reduces the risk associated with long term CDK4/6 inhibition. This cycling between a state of G1-arrest and a state of replication is not feasible in limited time-spaced, repeated IR exposures using longer acting CDK4/6 inhibitors such as PD0332991, as the lingering G1-arresting effects of the compound prohibit significant and meaningful reentry into the cell-cycle before the next IR exposure or delay reentry of the HSPCs from entering the cell cycle and reconstituting hematological cells following IR treatment cessation.

In one embodiment, the use of a CDK4/6 inhibitor described herein provides for a rapid, synchronous, reentry into the cell cycle by HSPCs so that the HSPCs return to pre-treatment baseline cell cycle activity within about 48 hours, within about 36 hours, within about 30 hours, within about 28 hours, within about 24 hours or less from IR cessation. In one embodiment, the use of a CDK4/6 inhibitor described herein provides for a rapid, synchronous, reentry into the cell cycle by HSPCs so that the HSPCs approach pre-treatment baseline cell cycle activity within less than 40 hours, about 36 hours, within about 30 hours, within about 28 hours, within about 24 hours or less from IR cessation. In one embodiment, the use of a CDK4/6 inhibitor described herein provides for a rapid, synchronous, reentry into the cell cycle by HSPCs so that the HSPCs return to pre-treatment baseline cell cycle activity within about 40 hours, within about 36 hours, within about 30 hours, within about 28 hours, within about 24 hours or less from the last CDk4/6 inhibitor administration. In one embodiment, the use of a CDK4/6 inhibitor described herein provides for a rapid, synchronous, reentry into the cell cycle by HSPCs so that the HSPCs approach pre-treatment baseline cell cycle activity within about within about 40 hours, within about 36 hours, within about 30 hours, within about 28 hours, within about 24 hours or less from the last CDk4/6 inhibitor administration. In one embodiment, the use of a CDK4/6 inhibitor described herein provides for a rapid, synchronous, reentry into the cell cycle by HSPCs so that the HSPCs approach pre-treatment baseline cell cycle activity within about 40 hours, within about 36 hours, within about 30 hours, within about 28 hours, within about 24 hours or less from the point in which the CDK4/6 inhibitor's concentration level in the subject's blood drops below a therapeutic effective concentration.

In one embodiment, the subject is exposed to IR at least 5 times a week, at least 4 times a week, at least 3 times a week, at least 2 times a week, at least 1 time a week, at least 3 times a month, at least 2 times a month, or at least 1 time a month, wherein the subject's HSPCs are G1 arrested during treatment and allowed to cycle in between IR exposure, for example during a treatment break. In one embodiment, the subject is undergoing 5 times a week IR exposure, wherein the subject's HSPCs are G1 arrested during the IR exposure and allowed to reenter the cell-cycle during the 2 day break, for example, over the weekend.

In one embodiment, using a CDK4/6 inhibitor described herein, the subject's HSPCs are arrested during the entirety of the IR exposure time-period for the weekly treatment, for example, during a 5 times/week IR regimen, the cells are arrested over the time period that is required to complete the IR exposure regimen for the week, and then allowed to recycle at the end of the regimen. In one embodiment, using a CDK4/6 inhibitor described herein, the subject's HSPCs are arrested during the entirety of the IR regimen, for example, in a 5 times a week IR regimen for 5 weeks, and rapidly reenter the cell-cycle following the completion of the IR regimen.

In one embodiment, the subject has been exposed to IR, and, using a CDK4/6 inhibitor described herein, the subject's HSPCs are placed in G1 arrest following exposure in order to mitigate DNA damage. In one embodiment, the CDK4/6 inhibitor is administered at least 1 hour, at least 2 hours, at least 3 hours, at least 4 hours, at least 5 hours, at least 6 hours, at least 7 hours, at least 8 hours, at least 10 hours, at least 12 hours, at least 14 hours, at least 16 hours, at least 18 hours, at least 20 hours, at least 24 hours or more post IR exposure. In one embodiment, the subject has been exposed to IR and is administered multiple CDK4/6 inhibitor doses at differing time points, for example, at 12 hours and 24 hours post IR exposure.

In some embodiments, the presently disclosed subject matter provides methods for protection of mammals from the acute and chronic toxic effects of ionizing radiation by forcing hematopoietic stem and progenitor cells (HSPCs) into a quiescent state by transient (e.g., over a less than less than about 40, 36, 30, 24 hour or less period) treatment with a CDK4/6 inhibitor selected from the group consisting of Formula I, Formula II, Formula III, Formula IV, or Formula V, or a pharmaceutically acceptable composition, salt, isotopic analog, or prodrug thereof. HSPCs recover from this period of transient quiescence, and then function normally after treatment with the inhibitor is stopped, and its intra-cellular effect dissipates. During the period of quiescence, the stem and progenitor cells are protected from the effects of ionizing radiation. The ability to protect stem/progenitor cells is desirable both in the treatment of cancer where patients are given high, repeated doses of ionizing radiation, and in environmental or occupational situations where individuals may be in danger of being exposed to large doses of radiation.

In some embodiments, the HSPCs can be arrested for longer periods, for example, over a period of hours, days, and/or weeks, through multiple, time separated administrations of a CDK4/6 inhibitor described herein. Because of the rapid and synchronous reentry into the cell cycle by HSPCs upon dissipation of the CDK4/6 inhibitors intra-cellular effects, the HSPCs are capable of reconstituting the cell lineages faster than CDK4/6 inhibitors with longer G1 arresting profiles, for example PD0332991.

In one embodiment of the invention, these improved CDK4/6 inhibitors can be administered in a concerted regimen with a blood growth factor agent. As such, in one embodiment, the use of the compounds and methods described herein is combined with the use of hematopoietic growth factors including, but not limited to, granulocyte colony stimulating factor (G-CSF, for example, sold as Neupogen (filgrastin), Neulasta (peg-filgrastin), or lenograstin), granulocyte-macrophage colony stimulating factor (GM-CSF, for example sold as molgramostim and sargramostim (Leukine)), M-CSF (macrophage colony stimulating factor), thrombopoietin (megakaryocyte growth development factor (MGDF), for example sold as Romiplostim and Eltrombopag) interleukin (IL)-12, interleukin-3, interleukin-11 (adipogenesis inhibiting factor or oprelvekin), SCF (stem cell factor, steel factor, kit-ligand, or KL) and erythropoietin (EPO), and their derivatives (sold as for example epoetin-α as Darbopoetin, Epocept, Nanokine, Epofit, Epogin, Eprex and Procrit; epoetin-β sold as for example NeoRecormon, Recormon and Micera), epoetin-delta (sold as for example Dynepo), epoetin omega (sold as for example Epomax), epoetin zeta (sold as for example Silapo and Reacrit) as well as for example Epocept, EPOTrust, Erypro Safe, Repoeitin, Vintor, Epofit, Erykine, Wepox, Espogen, Relipoeitin, Shanpoietin, Zyrop and EPIAO).

It has been recently been reported that some of the hematopoietic growth factors can have serious side effects. For example, the EPO family of therapeutics has been associated with arterial hypertension, cerebral convulsions, hypertensive encephalopathy, tumor progression thromboembolism, iron deficiency, influenza like syndromes and venous thrombosis. The G-CSF family of therapeutics has been associated with myelodysplasia and secondary leukemia, spleen enlargement and rupture, respiratory distress syndrome, allergic reactions and sickle cell complications.

By combining the administration of the improved very effective and selective CDK4/6 inhibitors and methods of the present invention with hematopoietic growth factors, it is possible for the health care practitioner to decrease the amount of the growth factor to minimize the unwanted adverse effects while achieving the therapeutic benefit. Thus, in this embodiment, the CDK4/CDK6 inhibitor allows the patient to receive some amount of the growth factor. The patient will not need as much hematopoietic growth factor because the hematopoietic cells will have been protected during the chemotherapy and not diminished to the extent without the CDK 4/6 inhibitor. Furthermore, by timing the administration of the growth factors, hematopoietic cells are not forced into replicating while harboring major DNA structural damage.

Several advantages can result from the radio-protective methods described herein using a selective CDK4/6 inhibitor described herein. The reduction in radio-toxicity afforded by the selective CDK4/6 inhibitors can allow for dose intensification (e.g., more therapy can be given in a fixed period of time) in medically related IR therapies, which will translate to better efficacy. Therefore, the presently disclosed methods can result in radio-therapy regimens that are less toxic and more effective. Also, in contrast to protective treatments with exogenous biological growth factors, the selective CDK4/6 inhibitors described herein are orally available small molecules, which can be formulated for administration via a number of different routes. When appropriate, such small molecules can be formulated for oral, topical, intranasal, inhalation, intravenous, intramuscular, or any other form of administration. Further, as opposed to biologics, stable small molecules can be more easily stockpiled and stored. Thus, the selective CDK4/6 inhibitor compounds can be more easily and cheaply kept on hand in emergency rooms where subjects of IR exposure can report or at sites where radiation exposure is particularly likely to occur: at nuclear power plants, on nuclear powered vessels, at military installations, near battlefields, etc.

CDK4/6 inhibitors useful in the methods described herein are selective CDK4/6 inhibitor compounds that selectively inhibit at least one of CDK4 and CDK6, or whose predominant mode of action is through inhibition of CDK4 and/or CDK6. In one embodiment, the selective CDK4/6 inhibitors have an IC₅₀ for CDK4 as measured in a CDK4/CycD1 IC₅₀ phosphorylation assay that is at least 1500 times or greater lower than the compound's IC₅₀, for CDK2 as measured in a CDK2/CycE IC₅₀ phosphorylation assay. In one embodiment, the CDK4/6 inhibitors are at least about 10 times or greater more potent (i.e., have an IC₅₀ in a CDK4/CycD1 phosphorylation assay that is at least 10 times or more lower) than PD0332991.

The use of a selective CDK4/6 inhibitor as described herein can induce selective G1 arrest in CDK4/6-dependent cells (e.g., as measured in a cell-based in vitro assay). In one embodiment, the CDK4/6 inhibitor is capable of increasing the percentage of CDK4/6-dependent cells in the G1 phase, while decreasing the percentage of CDK4/6-dependent cells in the G2/M phase and S phase. In one embodiment, the selective CDK4/6 inhibitor induces substantially pure (i.e., “clean”) G1 cell cycle arrest in the CDK4/6-dependent cells (e.g., wherein treatment with the selective CDK4/6 inhibitor induces cell cycle arrest such that the majority of cells are arrested in G1 as defined by standard methods (e.g. propidium iodide (PI) staining or others) with the population of cells in the G2/M and S phases combined being less than about 30%, about 25%, about 20%, about 15%, about 10%, about 5%, about 3% or less of the total cell population. Methods of assessing the cell phase of a population of cells are known in the art (see, for example, in U.S. Patent Application Publication No. 2002/0224522) and include cytometric analysis, microscopic analysis, gradient centrifugation, elutriation, fluorescence techniques including immunofluorescence, and combinations thereof. Cytometric techniques include exposing the cell to a labeling agent or stain, such as DNA-binding dyes, e.g., PI, and analyzing cellular DNA content by flow cytometry. Immunofluorescence techniques include detection of specific cell cycle indicators such as, for example, thymidine analogs (e.g., 5-bromo-2-deoxyuridine (BrdU) or an iododeoxyuridine), with fluorescent antibodies.

In some embodiments, the use of a selective CDK4/6 inhibitor described herein result in reduced or substantially free of off-target effects, particularly related to inhibition of kinases other than CDK4 and or CDK6 such as CDK2, as the selective CDK4/6 inhibitors described herein are poor inhibitors (e.g., >1 μM IC₅₀) of CDK2. Furthermore, because of the high selectivity for CDK4/6, the use of the compounds described herein should not induce cell cycle arrest in CDK4/6-independent cells. In addition, because of the short transient nature of the G1-arrest effect, HSPCs more quickly reenter the cell-cycle than, comparatively, use of PD0332991 provides, resulting in the reduced risk of hematological toxicity development during long term treatment regimens due to the ability of HSPCs to replicate between IR treatments.

In some embodiments, the use of a selective CDK4/6 inhibitor described herein reduces the risk of undesirable off-target effects including, but not limited to, long term toxicity, anti-oxidant effects, and estrogenic effects. Anti-oxidant effects can be determined by standard assays known in the art. For example, a compound with no significant anti-oxidant effects is a compound that does not significantly scavenge free-radicals, such as oxygen radicals. The anti-oxidant effects of a compound can be compared to a compound with known anti-oxidant activity, such as genistein. Thus, a compound with no significant anti-oxidant activity can be one that has less than about 2, 3, 5, 10, 30, or 100 fold anti-oxidant activity relative to genistein. Estrogenic activities can also be determined via known assays. For instance, a non-estrogenic compound is one that does not significantly bind and activate the estrogen receptor. A compound that is substantially free of estrogenic effects can be one that has less than about 2, 3, 5, 10, 20, or 100 fold estrogenic activity relative to a compound with estrogenic activity, e.g., genistein.

In some embodiments, the subject has been exposed to ionizing radiation, will be exposed to ionizing radiation, or is at risk of incurring exposure to ionizing radiation as the result of radiological agent exposure during warfare, a radiological terrorist attack, an industrial accident, or space travel. Subjects can further be exposed to, or be scheduled to be exposed to, ionizing radiation when undergoing therapeutic irradiation for the treatment of proliferative disorders. Such disorders include cancerous and non-cancer proliferative diseases. The compounds are effective in protecting healthy hematopoietic stem/progenitor cells during therapeutic irradiation of a broad range of tumor types, including but not limited to the following: breast, prostate, ovarian, skin, lung, colorectal, brain (i.e., glioma) and renal. Ideally, growth of the cancer being treated by IR should not be affected by the selective CDK 4/6 inhibitor. The potential sensitivity of certain tumors to CDK4/6 inhibition can be deduced based on tumor type and molecular genetics using standard techniques. Cancers that are not typically affected by the inhibition of CDK4/6 are those that can be characterized by one or more of the group including, but not limited to, increased activity of CDK1 or CDK2, loss or absence of retinoblastoma (Rb) tumor suppressor protein (Rb-null), high levels of MYC expression, increased cyclin E and increased cyclin A. Such cancers can include, but are not limited to, small cell lung cancer, retinoblastoma, HPV positive malignancies like cervical cancer and certain head and neck cancers, MYC amplified tumors such as certain classes of Rb-positive Burkitts Lymphoma, and triple negative breast cancer; certain classes of sarcoma, certain classes of non-small cell lung carcinoma, certain classes of melanoma, certain classes of pancreatic cancer, certain classes of leukemias, certain classes of lymphomas, certain classes of brain cancer, certain classes of colon cancer, certain classes of prostate cancer, certain classes of ovarian cancer, certain classes of uterine cancer, certain classes of thyroid and other endocrine tissue cancers, certain classes of salivary cancers, certain classes of thymic carcinomas, certain classes of kidney cancers, certain classes of bladder cancer and certain classes of testicular cancers.

The loss or absence of retinoblastoma (Rb) tumor suppressor protein (Rb-null) can be determined through any of the standard assays known to one of ordinary skill in the art, including but not limited to Western Blot, ELISA (enzyme linked immunoadsorbent assay), IHC (immunohistochemistry), and FACS (fluorescent activated cell sorting). The selection of the assay will depend upon the tissue, cell line or surrogate tissue sample that is utilized e.g., for example Western Blot and ELISA may be used with any or all types of tissues, cell lines or surrogate tissues, whereas the IHC method would be more appropriate wherein the tissue utilized in the methods of the present invention was a tumor biopsy. FACs analysis would be most applicable to samples that were single cell suspensions such as cell lines and isolated peripheral blood mononuclear cells. See for example, US 20070212736 “Functional Immunohistochemical Cell Cycle Analysis as a Prognostic Indicator for Cancer”.

Alternatively, molecular genetic testing may be used for determination of retinoblastoma gene status. Molecular genetic testing for retinoblastoma includes the following as described in Lohmann and Gallie “Retinoblastoma. Gene Reviews” (2010) http://www.ncbi.nlm.nih.gov/bookshelf/brIcgi?book=gene&part=retinoblastoma or Parsam et al. “A comprehensive, sensitive and economical approach for the detection of mutations in the RB 1 gene in retinoblastoma” Journal of Genetics, 88(4), 517-527 (2009).

Increased activity of CDK1 or CDK2, high levels of MYC expression, increased cyclin E and increased cyclin A can be determined through any of the standard assays known to one of ordinary skill in the art, including but not limited to Western Blot, ELISA (enzyme linked immunoadsorbent assay), IHC (immunohistochemistry), and FACS (fluorescent activated cell sorting). The selection of the assay will depend upon the tissue, cell line or surrogate tissue sample that is utilized e.g., for example Western Blot and ELISA may be used with any or all types of tissues, cell lines or surrogate tissues, whereas the IHC method would be more appropriate wherein the tissue utilized in the methods of the present invention was a tumor biopsy. FACs analysis would be most applicable to samples that were single cell suspensions such as cell lines and isolated peripheral blood mononuclear cells.

In some embodiments, the cancer a small cell lung cancer, retinoblastoma, and triple negative (ER/PR/Her2 negative) or “basal-like” breast cancer, which almost always inactivate the retinoblastoma tumor suppressor protein (Rb), and therefore do not require CDK4/6 activity to proliferate. Triple negative (basal-like) breast cancer is also almost always genetically or functionally Rb-null. Also, certain virally induced cancers (e.g. cervical cancer and subsets of Head and Neck cancer) express a viral protein (E7) which inactivates Rb making these tumors functionally Rb-null. Some lung cancers are also believed to be caused by HPV.

The selective CDK4/6 inhibitors described herein can also be used in protecting healthy CDK4/6-replication dependent cells during ionizing radiation of abnormal tissues in non-cancer proliferative diseases, including but not limited to the following: psoriasis, lupus, arthritis (notably rheumatoid arthritis), hemangiomatosis in infants, multiple sclerosis, myelodegenerative disease, neurofibromatosis, ganglioneuromatosis, keloid formation, Paget's Disease of the bone, fibrocystic disease of the breast, Peyronie's and Duputren's fibrosis, restenosis, and cirrhosis.

According to the present invention, therapeutic ionizing radiation can be administered to a subject on any schedule and in any dose consistent with the prescribed course of treatment, for example by administering a compound of Formula I, Formula II, Formula III, Formula IV or Formula V prior to or during the radiation. Preferably, administration of the inhibitor is timed such that maximal G1 arrest of the HSPCs, or a significant portion thereof, occurs at the time of the IR exposure. In certain embodiments, the CDK4/6 inhibitors described herein are administered so that a peak serum concentration for the inhibitor is reached at or near the time of IR exposure. If desired, multiple doses of the radioprotectant compound can be administered to the subject. Alternatively, the subject can be given a single dose of the inhibitor. The course of treatment differs from subject to subject, and those of ordinary skill in the art can readily determine the appropriate dose and schedule of therapeutic radiation in a given clinical situation.

### III. Synthesis of Select CDK4/6 Inhibitors

CDK4/6 inhibitors of the present invention can be synthesized according to the generalized Scheme 1 below. Specific synthesis and characterization of the substituted 2-aminopyrmidines useful for the synthesis of Formula III and Formula IV can be found in, for instance, WO2012/061156 (5-(4-isopropylpiperazin-1-yl)pyridine-2-amine and 5-(4-morpholino-1-piperidyl)pyridine-2-amine respectively). Formula I and Formula II can be synthesized according to Scheme 1 using the corresponding substituted 2-aminopyrimidines or as described in WO2012/061156.

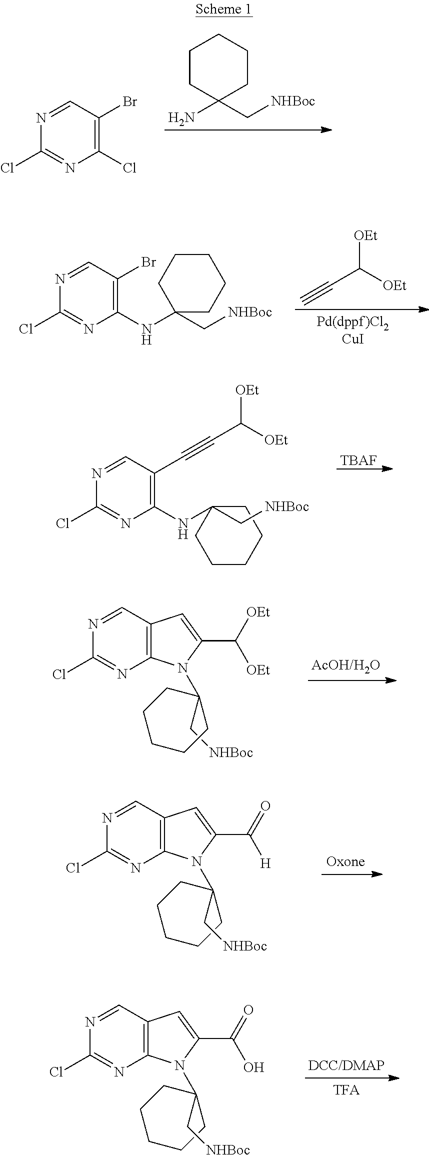

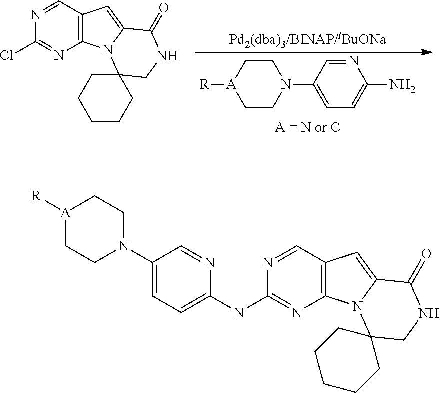

Formula I, II, III and IV as prepared above were characterized by mass spectrometry and NMR as shown below:

### Formula I

1H NMR (600 MHz, DMSO-d₆) δ ppm 1.47 (br. s., 6H) 1.72 (br. s., 2H) 1.92 (br. s., 2H) 2.77 (br. s., 3H) 3.18 (br. s., 2H) 3.46 (br. s., 2H) 3.63 (br. s., 2H) 3.66 (d, J=6.15 Hz, 2H) 3.80 (br. s., 2H) 7.25 (s, 1H) 7.63 (br. s., 2H) 7.94 (br. s., 1H) 8.10 (br. s., 1H) 8.39 (br. s., 1H) 9.08 (br. s., 1H) 11.59 (br. s., 1H). LCMS ESI (M+H) 447.

### Formula II

1H NMR (600 MHz, DMSO-d₆) δ ppm 0.82 (d, J=7.32 Hz, 2H) 1.08-1.37 (m, 3H) 1.38-1.64 (m, 2H) 1.71 (br. s., 1H) 1.91 (br. s., 1H) 2.80 (br. s., 1H) 3.12 (s, 1H) 3.41 (br. s., 4H) 3.65 (br. s., 4H) 4.09 (br. s., 1H) 7.26 (s, 1H) 7.52-7.74 (m, 2H) 7.94 (br. s., 1H) 8.13 (br. s., 1H) 8.40 (br. s., 1H) 9.09 (br. s., 1H) 9.62 (br. s., 1H) 11.71 (br. s., 1H). LCMS ESI (M+H) 433

### Formula III

1H NMR (600 MHz, DMSO-d₆) δ ppm 0.85 (br. s., 1H) 1.17-1.39 (m, 7H) 1.42-1.58 (m, 2H) 1.67-1.84 (m, 3H) 1.88-2.02 (m, 1H) 2.76-2.93 (m, 1H) 3.07-3.22 (m, 1H) 3.29-3.39 (m, 1H) 3.41-3.61 (m, 4H) 3.62-3.76 (m, 4H) 3.78-3.88 (m, 1H) 4.12 (br. s., 1H) 7.28 (s, 1H) 7.60-7.76 (m, 2H) 7.98 (s, 1H) 8.13 (br. s., 1H) 8.41 (s, 1H) 9.10 (br. s., 1H) 11.21 (br. s., 1H) 11.54 (s, 1H). LCMS ESI (M+H) 475

### Formula IV

1H NMR (600 MHz, DMSO-d₆) δ ppm 0.84 (t, J=7.61 Hz, 2H) 1.13-1.39 (m, 4H) 1.46 (d, J=14.05 Hz, 2H) 1.64-1.99 (m, 6H) 2.21 (br. s., 1H) 2.66-2.89 (m, 2H) 3.06 (br. s., 1H) 3.24-3.36 (m, 1H) 3.37-3.50 (m, 2H) 3.56-3.72 (m, 2H) 3.77-4.00 (m, 4H) 4.02-4.19 (m, 2H) 7.25 (s, 1H) 7.50-7.75 (m, 2H) 7.89 (d, J=2.93 Hz, 1H) 8.14 (d, J=7.32 Hz, 1H) 8.38 (br. s., 1H) 9.06 (s, 1H) 11.53 (br. s., 1H). LCMS ESI (M+H) 517

### V. Active Compounds, Salts and Formulations

As used herein, the term “active compound” refers to the selective CDK 4/6 inhibitor compounds described herein or a pharmaceutically acceptable salt or isotopic analog thereof. The active compound can be administered to the subject through any suitable approach. The amount and timing of active compound administered is dependent on the subject being treated, on the dosage of IR to which the subject is anticipated of being exposed to, on the time course of the IR exposure, on the manner of administration, on the pharmacokinetic properties of the particular active compound, and on the judgment of the prescribing physician. Thus, because of subject to subject variability, the dosages given below are a guideline and the physician can titrate doses of the compound to achieve the treatment that the physician considers appropriate for the subject. In considering the degree of treatment desired, the physician can balance a variety of factors such as age and weight of the subject, presence of preexisting disease, as well as presence of other diseases. Pharmaceutical formulations can be prepared for any desired route of administration including, but not limited to, oral, intravenous, or aerosol administration, as discussed in greater detail below.

The therapeutically effective dosage of any of the active compound described herein will be determined by the health care practitioner depending on the condition, size and age of the patient as well as the route of delivery. In one embodiment, a dosage from about 0.1 to about 200 mg/kg is administered, with all weights being calculated based upon the weight of the active compound, including the cases where a salt is employed. For example, a dosage can provide the amount of compound needed to provide a serum concentration of the active compound of up to between about 1 and 5, 10, 20, 30 or 40 μM. In some embodiments, a dosage from about 10 mg/kg to about 50 mg/kg can be employed for oral administration. Typically, a dosage from about 0.5 mg/kg to 5 mg/kg can be employed for intramuscular injection. In some embodiments, dosages can be from about 1 umol/kg to about 50 umol/kg, or, optionally, between about 22 umol/kg and about 33 umol/kg of the compound for intravenous or oral administration. An oral dosage form can include any appropriate amount of active material, including for example from 5 mg to, 50, 100, 200 or 500 mg per tablet or other solid dosage form.

In accordance with the presently disclosed methods, pharmaceutically active compounds as described herein can be administered orally as a solid or as a liquid, or can be administered intramuscularly, intravenously, or by inhalation as a solution, suspension, or emulsion. In some embodiments, the compounds or salts also can be administered by inhalation, intravenously, or intramuscularly as a liposomal suspension. When administered through inhalation the active compound or salt can be in the form of a plurality of solid particles or droplets having any desired particle size, and for example, from about 0.01, 0.1 or 0.5 to about 5, 10, 20 or more microns, and optionally from about 1 to about 2 microns. Compounds as disclosed in the present invention have demonstrated good pharmacokinetic and pharmacodynamics properties, for instance when administered by the oral or intravenous routes.

The pharmaceutical formulations can comprise an active compound described herein or a pharmaceutically acceptable salt thereof, in any pharmaceutically acceptable carrier. If a solution is desired, water is a carrier of choice for water-soluble compounds or salts. With respect to the water-soluble compounds or salts, an organic vehicle, such as glycerol, propylene glycol, polyethylene glycol, or mixtures thereof, can be suitable. In the latter instance, the organic vehicle can contain a substantial amount of water. The solution in either instance can then be sterilized in a suitable manner known to those in the art, and for illustration by filtration through a 0.22-micron filter. Subsequent to sterilization, the solution can be dispensed into appropriate receptacles, such as depyrogenated glass vials. The dispensing is optionally done by an aseptic method. Sterilized closures can then be placed on the vials and, if desired, the vial contents can be lyophilized.

In addition to the active compounds or their salts, the pharmaceutical formulations can contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the formulations can contain antimicrobial preservatives. Useful antimicrobial preservatives include methylparaben, propylparaben, and benzyl alcohol. An antimicrobial preservative is typically employed when the formulation is placed in a vial designed for multi-dose use. The pharmaceutical formulations described herein can be lyophilized using techniques well known in the art.

For oral administration a pharmaceutical composition can take the form of solutions, suspensions, tablets, pills, capsules, powders, and the like. Tablets containing various excipients such as sodium citrate, calcium carbonate and calcium phosphate may be employed along with various disintegrants such as starch (e.g., potato or tapioca starch) and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type may be also employed as fillers in soft and hard-filled gelatin capsules. Materials in this connection also include lactose or milk sugar as well as high molecular weight polyethylene glycols. When aqueous suspensions and/or elixirs are desired for oral administration, the compounds of the presently disclosed subject matter can be combined with various sweetening agents, flavoring agents, coloring agents, emulsifying agents and/or suspending agents, as well as such diluents as water, ethanol, propylene glycol, glycerin and various like combinations thereof.

In yet another embodiment of the subject matter described herein, there is provided an injectable, stable, sterile formulation comprising an active compound as described herein, or a salt thereof, in a unit dosage form in a sealed container. The compound or salt is provided in the form of a lyophilizate, which is capable of being reconstituted with a suitable pharmaceutically acceptable carrier to form a liquid formulation suitable for injection thereof into a subject. When the compound or salt is substantially water-insoluble, a sufficient amount of emulsifying agent, which is physiologically acceptable, can be employed in sufficient quantity to emulsify the compound or salt in an aqueous carrier. Particularly useful emulsifying agents include phosphatidyl cholines and lecithin.

Additional embodiments provided herein include liposomal formulations of the active compounds disclosed herein. The technology for forming liposomal suspensions is well known in the art. When the compound is an aqueous-soluble salt, using conventional liposome technology, the same can be incorporated into lipid vesicles. In such an instance, due to the water solubility of the active compound, the active compound can be substantially entrained within the hydrophilic center or core of the liposomes. The lipid layer employed can be of any conventional composition and can either contain cholesterol or can be cholesterol-free. When the active compound of interest is water-insoluble, again employing conventional liposome formation technology, the salt can be substantially entrained within the hydrophobic lipid bilayer that forms the structure of the liposome. In either instance, the liposomes that are produced can be reduced in size, as through the use of standard sonication and homogenization techniques. The liposomal formulations comprising the active compounds disclosed herein can be lyophilized to produce a lyophilizate, which can be reconstituted with a pharmaceutically acceptable carrier, such as water, to regenerate a liposomal suspension.

Pharmaceutical formulations also are provided which are suitable for administration as an aerosol by inhalation. These formulations comprise a solution or suspension of a desired compound described herein or a salt thereof, or a plurality of solid particles of the compound or salt. The desired formulation can be placed in a small chamber and nebulized. Nebulization can be accomplished by compressed air or by ultrasonic energy to form a plurality of liquid droplets or solid particles comprising the compounds or salts. The liquid droplets or solid particles may for example have a particle size in the range of about 0.5 to about 10 microns, and optionally from about 0.5 to about 5 microns. The solid particles can be obtained by processing the solid compound or a salt thereof, in any appropriate manner known in the art, such as by micronization. Optionally, the size of the solid particles or droplets can be from about 1 to about 2 microns. In this respect, commercial nebulizers are available to achieve this purpose. The compounds can be administered via an aerosol suspension of respirable particles in a manner set forth in U.S. Pat. No. 5,628,984, the disclosure of which is incorporated herein by reference in its entirety.

When the pharmaceutical formulation suitable for administration as an aerosol is in the form of a liquid, the formulation can comprise a water-soluble active compound in a carrier that comprises water. A surfactant can be present, which lowers the surface tension of the formulation sufficiently to result in the formation of droplets within the desired size range when subjected to nebulization.

The term “pharmaceutically acceptable salts” as used herein refers to those salts which are, within the scope of sound medical judgment, suitable for use in contact with subjects (e.g., human subjects) without undue toxicity, irritation, allergic response, and the like, commensurate with a reasonable benefit/risk ratio, and effective for their intended use, as well as the zwitterionic forms, where possible, of the compounds of the presently disclosed subject matter.

Thus, the term “salts” refers to the relatively non-toxic, inorganic and organic acid addition salts of compounds of the presently disclosed subject matter. These salts can be prepared in situ during the final isolation and purification of the compounds or by separately reacting the purified compound in its free base form with a suitable organic or inorganic acid and isolating the salt thus formed. In so far as the compounds of the presently disclosed subject matter are basic compounds, they are all capable of forming a wide variety of different salts with various inorganic and organic acids. Acid addition salts of the basic compounds are prepared by contacting the free base form with a sufficient amount of the desired acid to produce the salt in the conventional manner. The free base form can be regenerated by contacting the salt form with a base and isolating the free base in the conventional manner. The free base forms may differ from their respective salt forms in certain physical properties such as solubility in polar solvents.

Pharmaceutically acceptable base addition salts may be formed with metals or amines, such as alkali and alkaline earth metal hydroxides, or of organic amines. Examples of metals used as cations, include, but are not limited to, sodium, potassium, magnesium, calcium, and the like. Examples of suitable amines include, but are not limited to, N,N′-dibenzylethylenediamine, chloroprocaine, choline, diethanolamine, ethylenediamine, N-methylglucamine, and procaine.

The base addition salts of acidic compounds are prepared by contacting the free acid form with a sufficient amount of the desired base to produce the salt in the conventional manner. The free acid form can be regenerated by contacting the salt form with an acid and isolating the free acid in a conventional manner. The free acid forms may differ from their respective salt forms somewhat in certain physical properties such as solubility in polar solvents.

Salts can be prepared from inorganic acids sulfate, pyrosulfate, bisulfate, sulfite, bisulfite, nitrate, phosphate, monohydrogenphosphate, dihydrogenphosphate, metaphosphate, pyrophosphate, chloride, bromide, iodide such as hydrochloric, nitric, phosphoric, sulfuric, hydrobromic, hydriodic, phosphorus, and the like. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, laurylsulphonate and isethionate salts, and the like. Salts can also be prepared from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. and the like. Representative salts include acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Pharmaceutically acceptable salts can include cations based on the alkali and alkaline earth metals, such as sodium, lithium, potassium, calcium, magnesium and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. Also contemplated are the salts of amino acids such as arginate, gluconate, galacturonate, and the like. See, for example, Berge et al., J. Pharm. Sci., 1977, 66, 1-19, which is incorporated herein by reference.

### EXAMPLES

### Example 1

### CDK4/6 Inhibition In Vitro Assay

Selected compounds disclosed herein were tested in CDK4/cyclinD1, CDK6/CycD3 CDK2/CycA and CDK2/cyclinE kinase assays by Nanosyn (Santa Clara, Calif.) to determine their inhibitory effect on these CDKs. The assays were performed using microfluidic kinase detection technology (Caliper Assay Platform). The compounds were tested in 12-point dose-response format in singlicate at Km for ATP. Phosphoacceptor substrate peptide concentration used was 1 μM for all assays and Staurosporine was used as the reference compound for all assays. Specifics of each assay are as described below:

CDK2/CyclinA: Enzyme concentration: 0.2 nM; ATP concentration: 50 μM; Incubation time: 3 hr.

CDK2/CyclinE: Enzyme concentration: 0.28 nM; ATP concentration: 100 μM; Incubation time: 1 hr.

CDK4/CyclinD1: Enzyme concentration: 1 nM; ATP concentration: 200 μM; Incubation time: 10 hr.

CDK6/CyclinD3: Enzyme concentration: 1 nM; ATP concentration: 300 μM; Incubation time: 3 hr.

The results of the CDK6/CycD3 kinase assays, along with the CDK4/cyclinD1, CDK2/CycA and CDK2/cyclinE kinase assays, are shown for PD0332991 (Reference) and the Formulas I, II, III, and IV in Table 1. The IC₅₀ of 10 nM for CDK4/cyclinD1 and 10 uM for CDK12/CyclinE agrees well with previously published reports for PD0332991 (Fry et al. Molecular Cancer Therapeutics (2004) 3(11)1427-1437; Toogood et al. Journal of Medicinal Chemistry (2005) 48, 2388-2406). Formulas I, II, III, and IV are more potent (lower IC₅₀) with respect to the reference compound (PD0332991) and demonstrate a higher fold selectivity with respect to the reference compound (CDK2/CycE IC₅₀ divided by CDK4/CycD1 IC₅₀).

TABLE 1
Inhibition of CDK kinases by Formulas I, II, III, and IV

| | | CDK2/ | Fold | CDK2/ | CDK6/ |
|---|---|---|---|---|---|
| | CDK4/CycD1 | CycE | Selectivity | CycA | CycD3 |
| Formula | IC₅₀ (nM) | IC₅₀ (uM) | CDK2/CDK4 | IC₅₀ (uM) | IC₅₀ (nM) |
| PD0332991 | 10 | 10 | 1000 | Not | Not |
| Reference | | | | determined | determined |
| Formula I | 0.821 | 1.66 | 2022 | 1.67 | 5.64 |
| Formula II | 0.627 | 1.08 | 1722 | 3.03 | 4.38 |
| Formula III | 1.060 | 3.58 | 3377 | 1.51 | 4.70 |
| Formula IV | 0.655 | 1.46 | 2229 | .857 | 5.99 |

To further characterize its kinase activity, Formula I was screened against 456 (395 non-mutant) kinases using DiscoveRx's KINOMEscan™ profiling service. The compound was screened using a single concentration of 1000 nM (>1000 times the IC50 on CDK4). Results from this screen confirmed the high potency against CDK4 and high selectivity versus CDK2. Additionally, the kinome profiling showed that Formula I was relatively selective for CDK4 and CDK6 compared to the other kinases tested. Specifically, when using an inhibitory threshold of 65%, 90%, or 99%, Formula I inhibited 92 (23.3%), 31 (7.8%) or 6 (1.5%) of 395 non-mutant kinases respectively.

### Example 2

### G1 Arrest (Cellular G1 and S-Phase) Assay

For determination of cellular fractions in various stages of the cell cycle following various treatments, HS68 cells (human skin fibroblast cell line (Rb-positive)) were stained with propidium iodide staining solution and run on Dako Cyan Flow Cytometer. The fraction of cells in G0-G1 DNA cell cycle versus the fraction in S-phase DNA cell cycle was determined using FlowJo 7.2 0.2 analysis.

Formulas I, II, III, and IV were tested for their ability to arrest HS68 cells at the G1 phase of the cell cycle. From the results of the cellular G1 arrest assay, the range of the inhibitory EC₅₀ values necessary for G1 arrest of HS68 cells was from 25 nM to 100 nM (see column titled “Cellular G1 Arrest EC₅₀” in Table 2).

### Example 3

### Cell Cycle Arrest by Formula I in CDK4/6-Dependent Cells

To test the ability of CDK4/6 inhibitors to induce a clean G1-arrest, a cell based screening method was used consisting of two CDK4/6-dependent cell lines (tHS68 and WM2664; Rb-positive) and one CDK4/6-independent (A2058; Rb-negative) cell line. Twenty-four hours after plating, each cell line was treated with Formula I in a dose dependent manner for 24 hours. At the conclusion of the experiment, cells were harvested, fixed, and stained with propidium iodide (a DNA intercalator), which fluoresces strongly red (emission maximum 637 nm) when excited by 488 nm light. Samples were run on Dako Cyan flow cytometer and >10,000 events were collected for each sample. Data were analyzed using FlowJo 2.2 software developed by TreeStar, Inc.

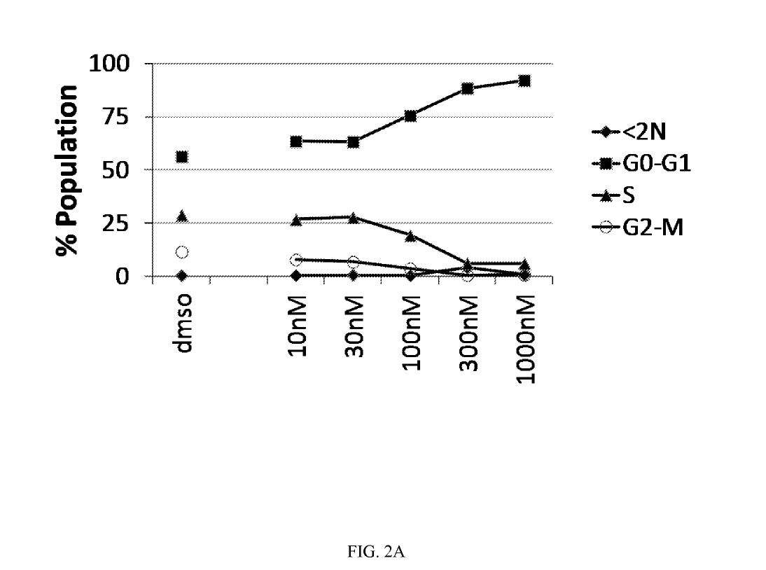

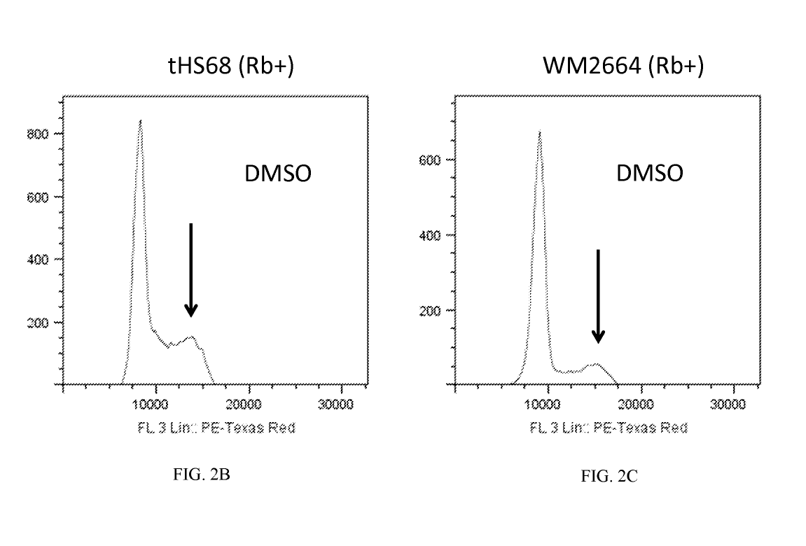

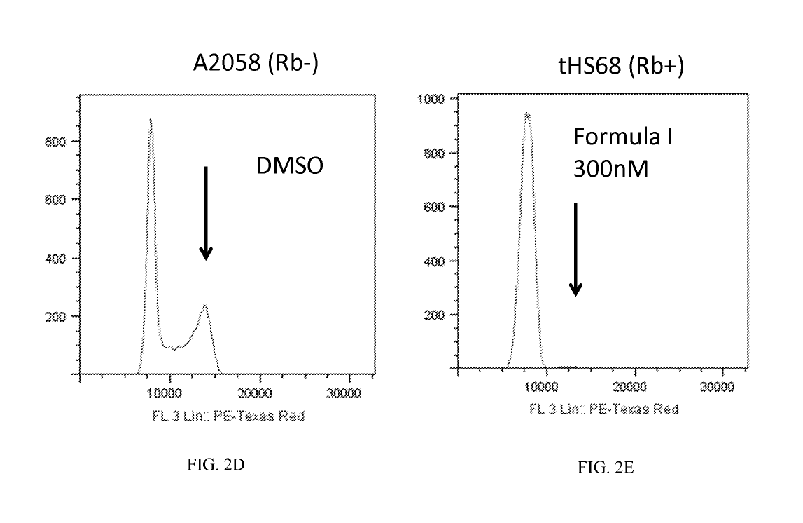

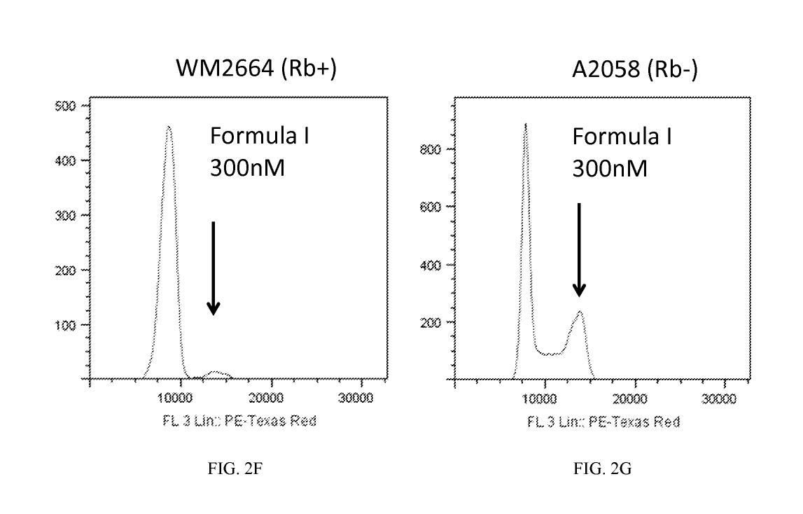

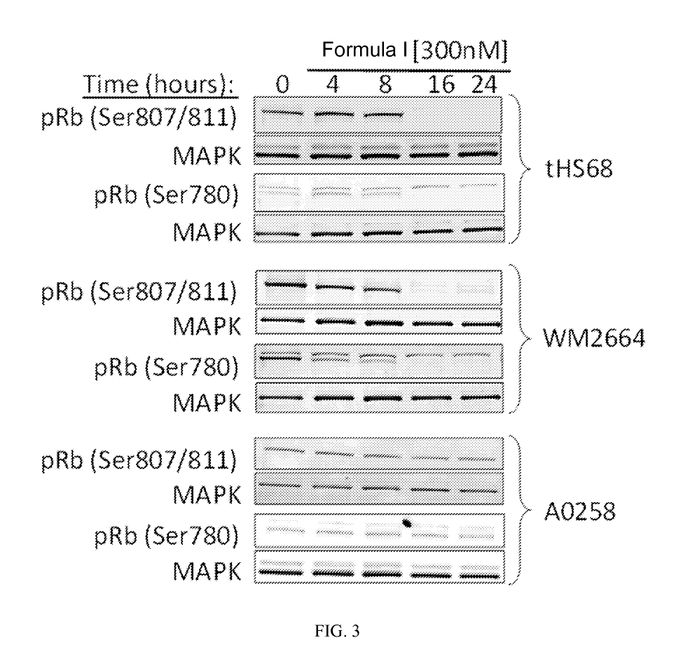

In FIG. 2A, results show that Formula I induces a robust G1 cell cycle arrest, as nearly all cells are found in the G0-G1 phase upon treatment with increasing amounts of Formula I. In FIG. 2A, the results show that in CDK4/6-dependent cell lines, Formula I induced a robust G1 cell cycle arrest with an EC₅₀ of 80 nM in tHS68 cells with a corresponding reduction in S-phase ranging from 28% at baseline to 6% at the highest concentration shown. Upon treatment with Formula I (300 nM), there was a similar reduction in the S-phase population and an increase in G1-arrested cells in both CDK4/6-dependent cell lines (tHS68 (Compare FIGS. 2B and 2E) and WM2664 (Compare FIGS. 2C and 2F)), but not in the CDK4/6-independent (A2058; Compare FIGS. 2D and 2G) cell line. The CDK4/6-independent cell line shows no effect in the presence of inhibitor.

### Example 4

### Formula I Inhibits Phosphorylation of RB

The CDK4/6-cyclin D complex is essential for progression from G1 to the S-phase of the DNA cell cycle. This complex phosphorylates the retinoblastoma tumor suppressor protein (Rb). To demonstrate the impact of CDK4/6 inhibition on Rb phosphorylation (pRb), Formula I was exposed to three cell lines, two CDK4/6 dependent (tHS68, WM2664; Rb-positive) and one CDK4/6 independent (A2058; Rb-negative). Twenty four hours after seeding, cells were treated with Formula I at 300 nM final concentration for 4, 8, 16, and 24 hours. Samples were lysed and protein was assayed by western blot analysis. Rb phosphorylation was measured at two sites targeted by the CDK4/6-cyclin D complex, Ser780 and Ser807/811 using species specific antibodies. Results demonstrate that Formula I blocks Rb phosphorylation in Rb-dependent cell lines by 16 hours post exposure, while having no effect on Rb-independent cells (FIG. 3).

### Example 5

### Small Cell Lung Cancer (SCLC) Cells are Resistant to CDK4/6 Inhibitors

The retinoblastoma (RB) tumor suppressor is a major negative cell cycle regulator that is inactivated in approximately 11% of all human cancers. Functional loss of RB is an obligate event in small cell lung cancer (SCLC) development. In RB competent tumors, activated CDK2/4/6 promote G1 to S phase traversal by phosphorylating and inactivating RB (and related family members). Conversely, cancers with RB deletion or inactivation do not require CDK4/6 activity for cell cycle progression. Since inactivation of RB is an obligate event in SCLC development, this tumor type is highly resistant to CDK4/6 inhibitors and co-administration of CDK4/6 inhibitors with DNA damaging chemotherapeutic agents such as those used in SCLC should not antagonize the efficacy of such agents.

Several compounds (PD0332991, Formula III, and Formula I) were tested for their ability to block cell proliferation in a panel of SCLC cell lines with known genetic loss of RB. SCLC cells were treated with DMSO or the indicated CDK4/6 inhibitor for 24 hours. The effect of CDK4/6 inhibition on proliferation was measured by EdU incorporation. An RB-intact, CDK4/6-dependent cell line (WM2664 or tHS68) and a panel of RB-negative SCLC cell lines (H69, H82, H209, H345, NCI417, or SHP-77) were analyzed for growth inhibition by the various CDK4/6 inhibitors.

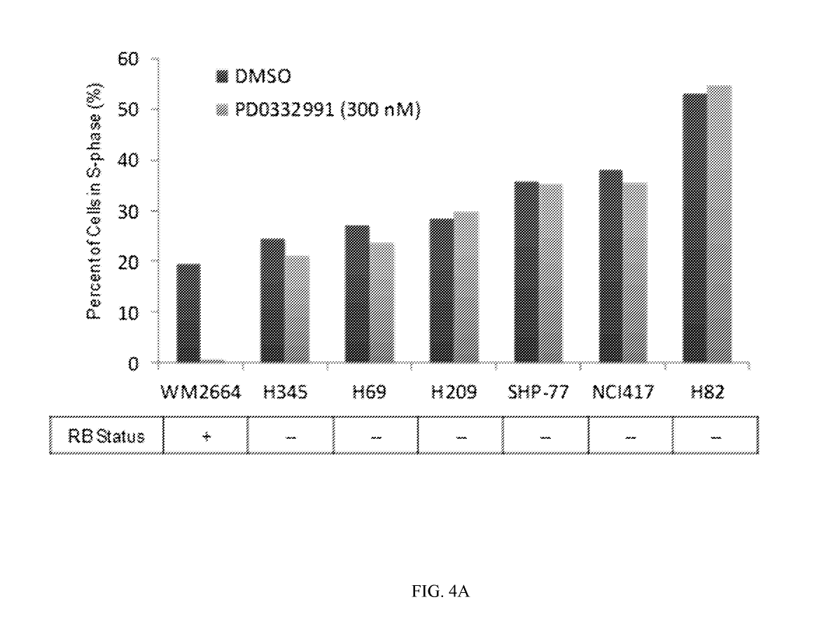

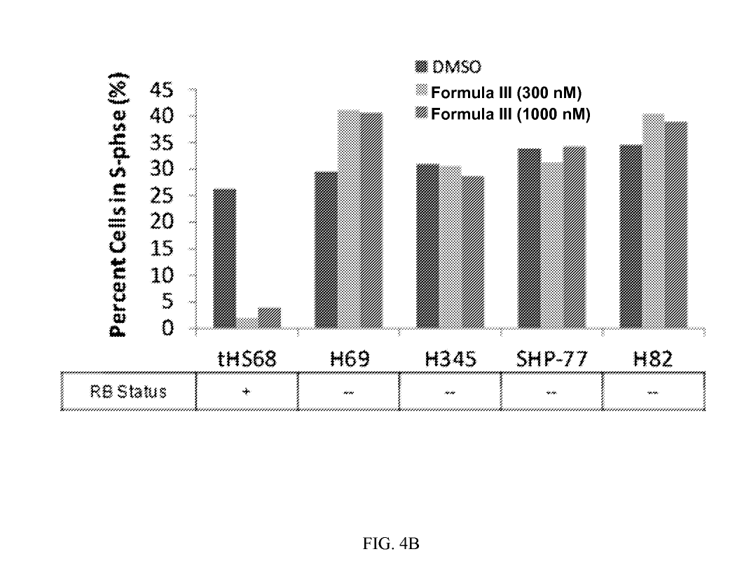

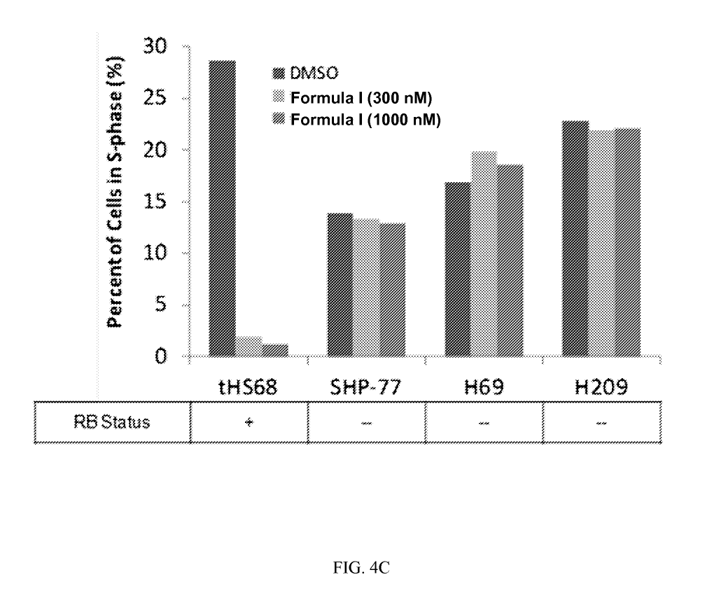

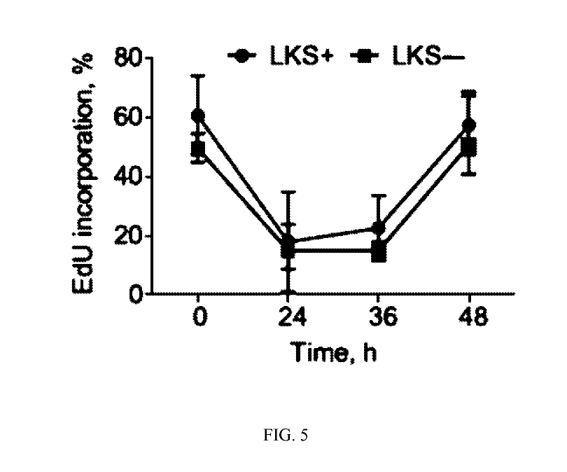

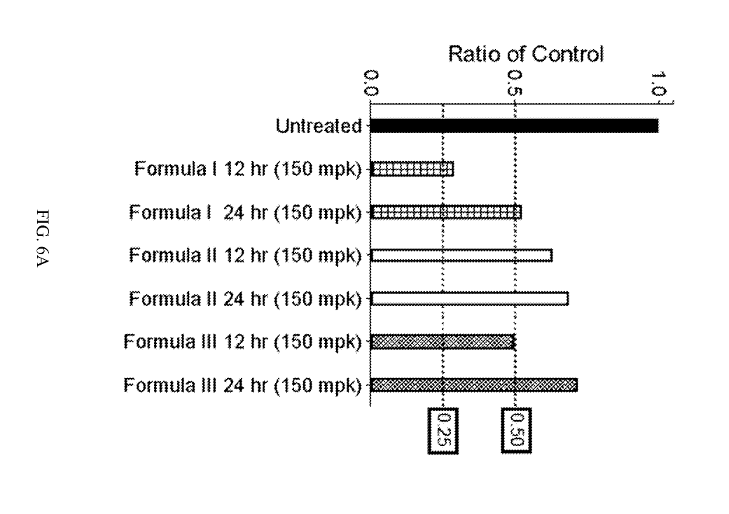

As shown in FIG. 4, Rb-negative SCLC cells are resistant to CDK4/6 inhibition. In FIG. 4A, PD0332991 inhibits the Rb-positive cell line (WM2664), but does not affect the growth of the Rb-negative small cell lung cancer cell lines (H345, H69, H209, SHP-77, NCI417, and H82). In FIG. 4B, Formula III inhibits the Rb-positive cell line (tHS68), but does not affect the growth of the Rb-negative cell lines (H345, H69, SHP-77, and H82). In FIG. 4C, Formula I inhibits the Rb-positive cell line (tHS68), but does not affect the growth of the Rb-negative cell lines (H69, SHP-77, and H209). This analysis demonstrated that RB-null SCLC cell lines were resistant to CDK4/6 inhibition, as no change in the percent of cells in S-phase were seen upon treatment with any of the CDK4/6 inhibitors tested, including Formula I and Formula III, while the RB-proficient cell line in each experiment was highly sensitive to CDK4/6 inhibition with almost no cells remaining in S-phase after 24 hours of treatment.

### Example 6

### Rb-Negative Cancer Cells are Resistant to Described CDK4/6 Inhibitors

Cellular proliferation assays were conducted using the following Rb-negative cancer cell lines: H69 (human small cell lung cancer—Rb-negative) cells or A2058 (human metastatic melanoma cells—Rb-negative). These cells were seeded in Costar (Tewksbury, Mass.) 3093 96 well tissue culture treated white walled/clear bottom plates. Cells were treated Formulas I, II, III, or IV as nine point dose response dilution series from 10 uM to 1 nM. Cells were exposed to compounds and then cell viability was determined after either four (H69) or six (A2058) days as indicated using the CellTiter-Glo® luminescent cell viability assay (CTG; Promega, Madison, Wis., United States of America) following the manufacturer's recommendations. Plates were read on BioTek (Winooski, Vt.) Syngergy2 multi-mode plate reader. The Relative Light Units (RLU) were plotted as a result of variable molar concentration and data was analyzed using Graphpad (LaJolla, Calif.) Prism 5 statistical software to determine the EC₅₀ for each compound.

Select compounds disclosed herein were evaluated against a small cell lung cancer cell line (H69) and a human metastatic melanoma cell line (A2058), two Rb-deficient (Rb-negative) cell lines. The results of these cellular inhibition assays are shown in Table 2. The range of the inhibitory EC₅₀ values necessary for inhibition of H69 small cell lung cancer cells was 2920 nM to >3000 nM. The range of the inhibitory EC₅₀ values necessary for inhibition of A2058 malignant melanoma cell proliferation was 2610 nM to >3000 nM. In contrast to the significant inhibition seen on Rb-positive cell lines, it was found that the compounds tested were not significantly effective at inhibiting proliferation of the small cell lung cancer or melanoma cells.

TABLE 2
Resistance of Rb-Negative Cancer Cells to CDK4/6 Inhibitors

| | | H69 | |
|---|---|---|---|
| | Cellular G1 | Cellular | A2058 |
| | Arrest EC₅₀ | EC₅₀ | Cellular |
| Structure | [nM] | [nM] | EC₅₀ [nM] |
| Formula I | 100 | >3000 | >3000 |
| Formula II | 100 | >3000 | 2610 |
| Formula III | 80 | 2920 | 2691 |
| Formula IV | 25 | >3000 | >3000 |

### Example 7

### HSPC Growth Suppression Studies

The effect of PD0332991 on HSPCs has been previously demonstrated. FIG. 5 shows the EdU incorporation of mice HSPC and myeloid progenitor cells following a single dose of 150 mg/kg PD0332991 by oral gavage to assess the temporal effect of transient CDK4/6 inhibition on bone marrow arrest as reported in Roberts et al. Multiple Roles of Cyclin-Dependent Kinase 4/6 Inhibitors in Cancer Therapy. JCNI 2012; 104(6):476-487. As can be seen in FIG. 5, a single oral dose of PD0332991 results in a sustained reduction in HSPC (LKS+) and myeloid progenitor cells (LKS−) for greater than 36 hours. Not until 48 hours post oral dosing do HSPC and myeloid progenitor cells return to baseline cell division.

### Example 8

### Bone Marrow Proliferation as Evaluated Using EdU Incorporation and Flow Cytometry Analysis

For HSPC proliferation experiments, young adult female FVB/N mice were treated with a single dose as indicated of Formula I, Formula II, Formula III or PD0332991 by oral gavage. Mice were then sacrificed at the indicated times (0, 12, 24, 36, or 48 hours following compound administration), and bone marrow was harvested (n=3 mice per time point), as previously described (Johnson et al. J. Clin. Invest. (2010) 120(7), 2528-2536). Four hours before the bone marrow was harvested, mice were treated with 100 μg of EdU by intraperitoneal injection (Invitrogen). Bone marrow mononuclear cells were harvested and immunophenotyped using previously described methods and percent EdU positive cells were then determined (Johnson et al. J. Clin. Invest. (2010) 120(7), 2528-2536). In brief, HSPCs were identified by expression of lineage markers (Lin−), Scal (S+), and c-Kit (K+).

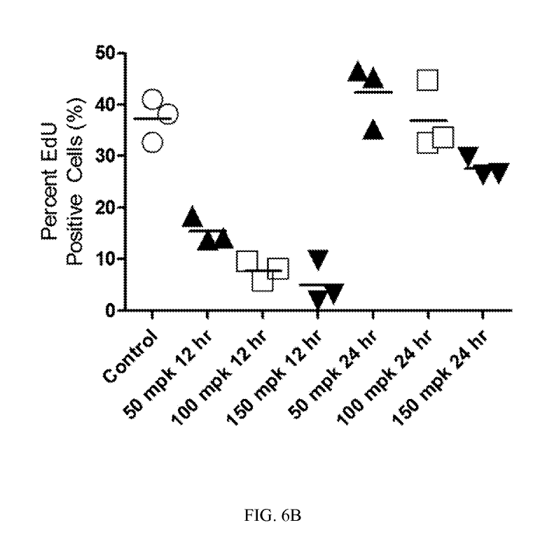

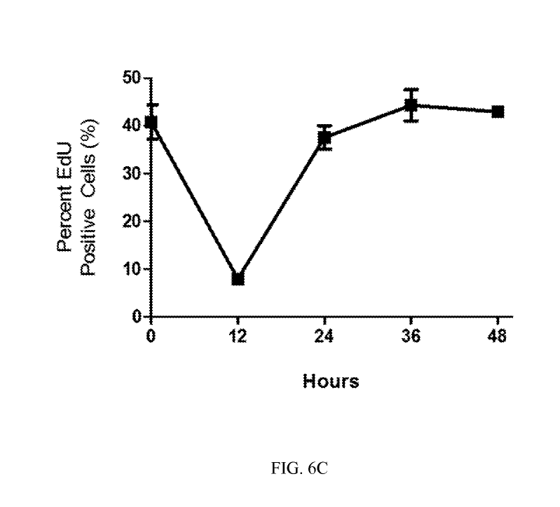

Analysis in mice determined that Formula I, Formula II, Formula III demonstrated dose dependent, transient, and reversible G1-arrest of bone marrow stem cells (HSPC) (FIG. 6). Six mice per group were dosed by oral gavage at 150 mg/kg of Formula I, Formula II, Formula III, or vehicle only. Four hours before animals were sacrificed and the bone marrow was harvested, mice were treated with 100 μg of EdU by intraperitoneal injection. Three mice per group were sacrificed at 12 hours and the remaining three animals per group were sacrificed at 24 hours. Results are shown in FIG. 6A as the ratio of EdU positive cells for treated animals at 12 or 24 hour time points compared to control. Formula I and GG demonstrated a reduction in EdU incorporation at 12 hours which was starting to return to normal at 24 hours. Formula II also demonstrated some reduction at 12 hours and started to return to baseline at 24 hours despite the fact that oral bioavailability of Formula II is low.

Further experiments were completed with Formula I examining dose response and longer periods of Formula I treatment. Formula I was dosed by oral gavage at 50, 100 or 150 mg/kg and EdU incorporation into bone marrow was determined at 12 and 24 hours as described above. Alternatively, Formula I was dosed by oral gavage at 150 mg/kg and EdU incorporation into bone marrow was determined at 12, 24, 36 and 48 hours. As can be seen in FIGS. 6B and 5C, and similar to the cellular washout experiments, bone marrow cells, and in particular HSPCs were returning to normal cell division as determined by EdU incorporation in 24 hours following oral gavage at a number of doses. The 150 mg/kg oral dose of Formula I in FIG. 6C can be compared directly to the results of the same dose of PD0332991 shown in FIG. 5 where cells were still non-dividing (as determined by low EdU incorporation) at 24 and 36 hours, only returning to normal values at 48 hours.

### Example 9

### HSPC Growth Suppression Studies Comparing Formula I and PD0332991

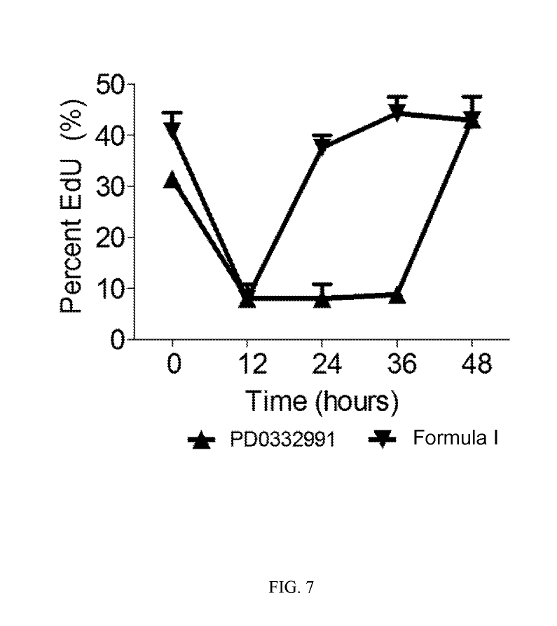

FIG. 7 is a graph of the percentage of EdU positive HSPC cells for mice treated with either PD0332991 (triangles) or Formula I (upside down triangles) v. time after administration (hours) of the compound. Both compounds were administered at 150 mg/kg by oral gavage. One hour prior to harvesting bone marrow, EdU was IP injected to label cycling cells. Bone marrow was harvested at 12, 24, 36, and 48 hours after Formula I treatment and the percentage of EdU positive HSPC cells was determined at each time point.

As seen in FIG. 7, a single oral dose of PD0332991 results in a sustained reduction in HSPCs for greater than 36 hours. In contrast, a single oral dose of Formula I results in an initial reduction of HSPC proliferation at 12 hours, but proliferation of HSPCs resumes by 24 hours after dosage of Formula I.

### Example 10

### Cellular Wash-Out Experiment

HS68 cells were seeded out at 40,000 cells/well in 60 mm dish on day 1 in DMEM containing 10% fetal bovine serum, 100 U/ml penicillin/streptomycin and 1× Glutamax (Invitrogen) as described (Brookes et al. EMBO J, 21(12)2936-2945 (2002) and Ruas et al. Mol Cell Biol, 27(12)4273-4282 (2007)). 24 hrs post seeding, cells are treated with Formula I, Formula II, Formula III, Formula IV, PD0332991, or DMSO vehicle alone at 300 nM final concentration of test compounds. On day 3, one set of treated cell samples were harvested in triplicate (0 Hour sample). Remaining cells were washed two times in PBS-CMF and returned to culture media lacking test compound. Sets of samples were harvested in triplicate at 24, 40, and 48 hours.

Alternatively, the same experiment was done using normal Renal Proximal Tubule Epithelial Cells (Rb-positive) obtained from American Type Culture Collection (ATCC, Manassas, Va.). Cells were grown in an incubator at 37° C. in a humidified atmosphere of 5% CO2 in Renal Epithelial Cell Basal Media (ATCC) supplemented with Renal Epithelial Cell Growth Kit (ATCC) in 37° C. humidified incubator.

Upon harvesting cells, samples were stained with propidium iodide staining solution and samples run on Dako Cyan Flow Cytometer. The fraction of cells in G0-G1 DNA cell cycle versus the fraction in S-phase DNA cell cycle was determined using FlowJo 7.2 0.2 analysis.

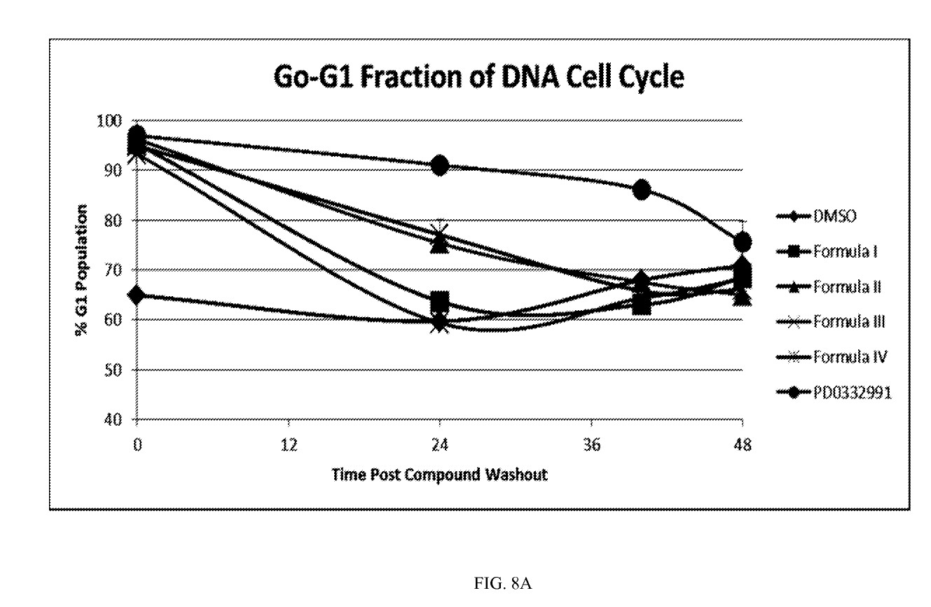

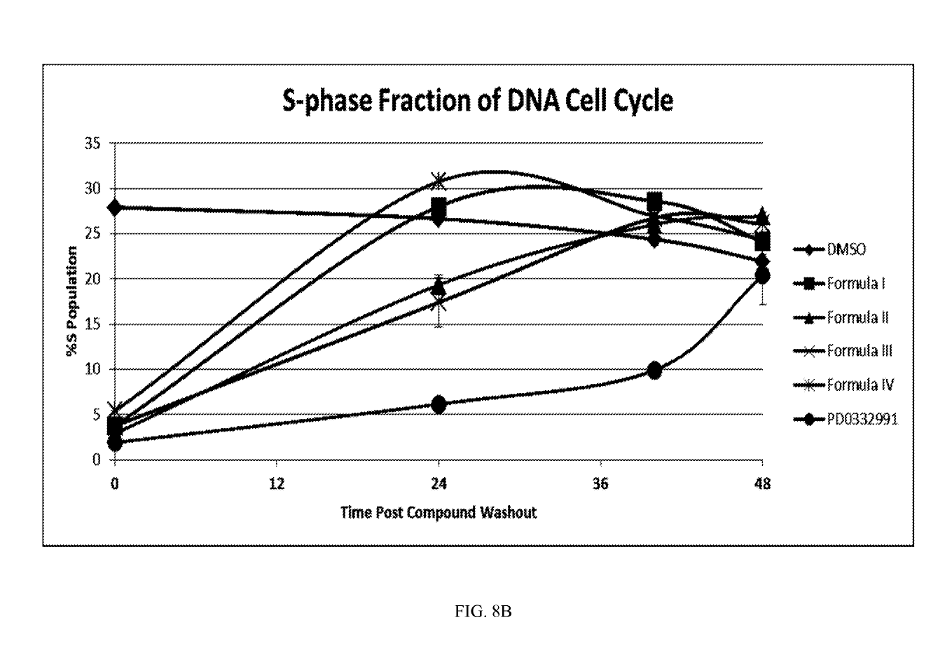

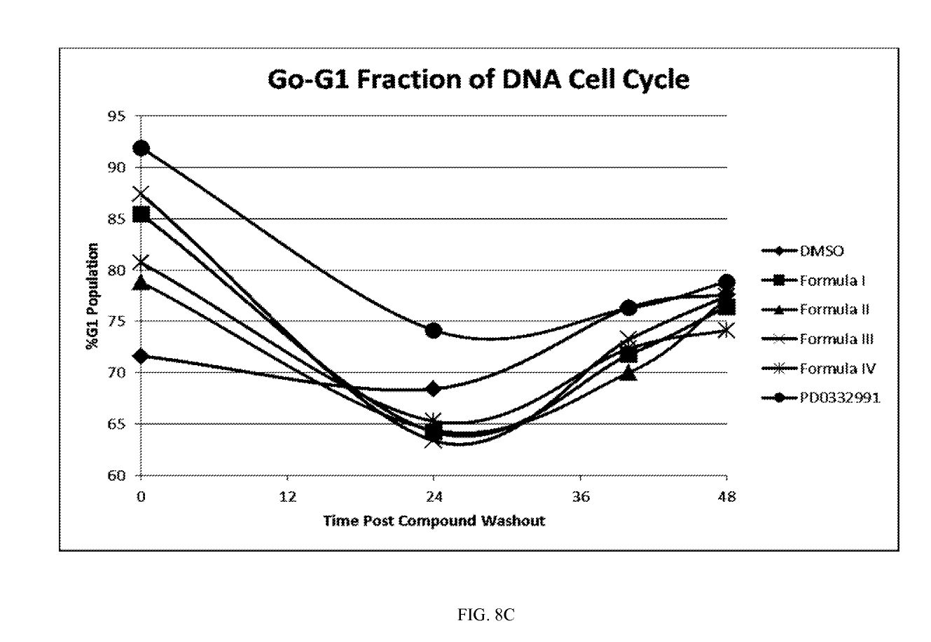

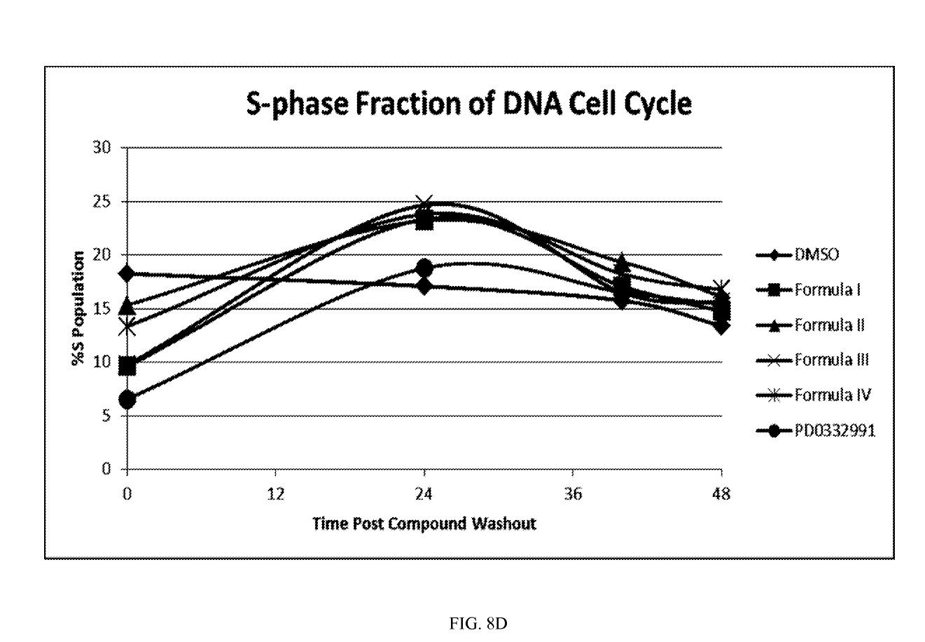

FIG. 8 shows cellular wash-out experiments which demonstrate the inhibitor compounds of the present invention have a short, transient G1-arresting effect in different cell types. Formulas I, II, III, and IV were compared to PD0332991 in either human fibroblast cells (Rb-positive) (FIGS. 8A & 8B) or human renal proximal tubule epithelial cells (Rb-positive) (FIGS. 8C & 8D) and the effect on cell cycle following washing out of the compounds was determined at 24, 36, 40, and 48 hours.

As shown in FIG. 8 and similar to results in vivo as shown in FIG. 5, PD0332991 required greater than 48 hours post wash out for cells to return to normal baseline cell division. This is seen in FIG. 8A and FIG. 8B as values equivalent to those for the DMSO control for either the G0-G1 fraction or the S-phase of cell division, respectively, were obtained. In contrast, HS68 cells treated with compounds of the present invention returned to normal baseline cell division in as little as 24 hours or 40 hours, distinct from PD0332991 at these same time points. The results using human renal proximal tubule epithelial cells (FIGS. 8C & 8D) also show that PD0332991-treated cells took significantly longer to return to baseline levels of cell division as compared to cells treated with Formulas I, II, III, and IV.

### Example 11

### Pharmacokinetic and Pharmacodynamic Properties of Anti-Neoplastic Compounds

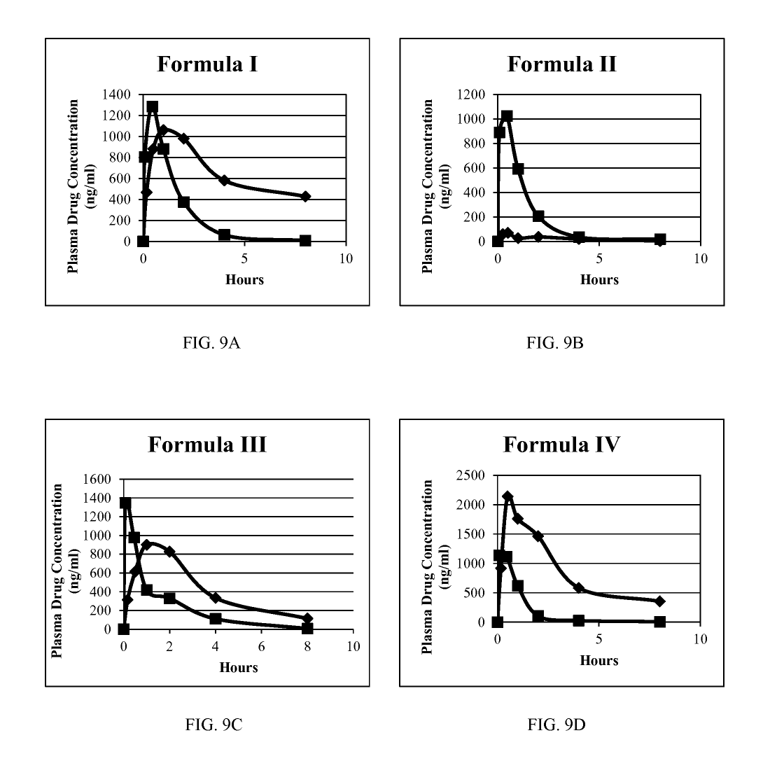

Compounds of the present invention demonstrate good pharmacokinetic and pharmacodynamic properties. Formulas I, II, III, and IV were dosed to mice at 30 mg/kg by oral gavage or 10 mg/kg by intravenous injection. Blood samples were taken at 0, 0.25, 0.5, 1.0, 2.0, 4.0, and 8.0 hours post dosing and the plasma concentration of Formula I, Q, GG, or U were determined by HPLC. Formulas I, III, and IV were demonstrated to have excellent oral pharmacokinetic and pharmacodynamic properties as shown in Table 3. This includes very high oral bioavailability (F(%)) of 52% to 80% and a plasma half-life of 3 to 5 hours following oral administration. Formulas I, II, III, and IV were demonstrated to have excellent pharmacokinetic and pharmacodynamic properties when delivered by intravenous administration. Representative IV and oral PK curves for all four compounds are shown in FIG. 9.

TABLE 3
Pharmacokinetic and pharmacodynamic properties of Formulas

| Mouse PK | Formula I | Formula II | Formula III | Formula IV |
|---|---|---|---|---|
| CL (mL/min/kg) | 35 | 44 | 82 | 52 |
| Vss (L/kg) | 2.7 | 5.2 | 7.5 | 3.4 |
| t_{1/2} (h) p.o. | 5 | 0.8 | 3.5 | 3 |
| AUC_{0-inf} (uM * h) | 1.3 | 0.95 | 1.1 | 0.76 |
| i.v. | | | | |
| AUC (uM * h) p.o. | 2.9 | 0.15 | 1.9 | 3.3 |
| Cₘₐₓ (uM) p.o. | 2.5 | 0.16 | 1.9 | 4.2 |
| Tₘₐₓ (h) p.o. | 1 | 0.5 | 1 | 0.5 |
| F (%) | 80 | 2 | 52 | 67 |

### Example 12

### Metabolic Stability

The metabolic stability of Formula I in comparison to PD0332991 was determined in human, dog, rat, monkey, and mouse liver microsomes. Human, mouse, and dog liver microsomes were purchased from Xenotech, and Sprague-Dawley rat liver microsomes were prepared by Absorption Systems. The reaction mixture comprising 0.5 mg/mL of liver microsomes, 100 mM of potassium phosphate, pH 7.4, 5 mM of magnesium chloride, and 1 uM of test compound was prepared. The test compound was added into the reaction mixture at a final concentration of 1 uM. An aliquot of the reaction mixture (without cofactor) was incubated in shaking water bath at 37 deg. C for 3 minutes. The control compound, testosterone, was run simultaneously with the test compound in a separate reaction. The reaction was initiated by the addition of cofactor (NADPH), and the mixture was then incubated in a shaking water bath at 37 deg. C. Aliquots (100 μL) were withdrawn at 0, 10, 20, 30, and 60 minutes for the test compound and 0, 10, 30, and 60 minutes for testosterone. Test compound samples were immediately combined with 100 μL of ice-cold acetonitrile containing internal standard to terminate the reaction. Testosterone samples were immediately combined with 800 μL of ice cold 50/50 acetonitrile/dH2O containing 0.1% formic acid and internal standard to terminate the reaction. The samples were assayed using a validated LC-MS/MS method. Test compound samples were analyzed using the Orbitrap high resolution mass spectrometer to quantify the disappearance of parent test compound and detect the appearance of metabolites. The peak area response ration (PARR) to internal standard was compared to the PARR at time 0 to determine the percent of test compound or positive control remaining at time-point. Half-lives were calculated using GraphPad software, fitting to a single-phase exponential decay equation.

Half-life was calculated based on t1/2=0.693k, where k is the elimination rate constant based on the slope plot of natural logarithm percent remaining versus incubation time. When calculated half-life was longer than the duration of the experiment, the half-life was expressed as >the longest incubation time. The calculated half-life is also listed in parentheses. If the calculated half-life is >2× the duration of the experiment, no half-life was reported. The timely resumption of cellular proliferation is necessary for tissue repair, and therefore an overly long period of arrest is undesirable in healthy cells such as HSPCs. The characteristics of a CDK4/6 inhibitor that dictate its arresting duration are its pharmacokinetic (PK) and enzymatic half-lives. Once initiated, a G1-arrest in vivo will be maintained as long as circulating compound remains at an inhibitory level, and as long as the compound engages the enzyme. PD032991, for example, possesses an overall long PK half-life and a fairly slow enzymatic off-rate. In humans, PD0332991 exhibits a PK half-life of 27 hours (see Schwartz, G K et al. (2011) BJC, 104:1862-1868). In humans, a single administration of PD0332991 produces a cell cycle arrest of HSPC lasting approximately one week. This reflects the 6 days to clear the compound (5 half-lives×27 hour half-life), as well as an additional 1.5 to 2 days of inhibition of enzymatic CDK4/6 function. This calculation suggests that it takes a total of 7+ days for normal bone marrow function to return, during which time new blood production is reduced. These observations may explain the severe granulocytopenia seen with PD0332991 in the clinic.

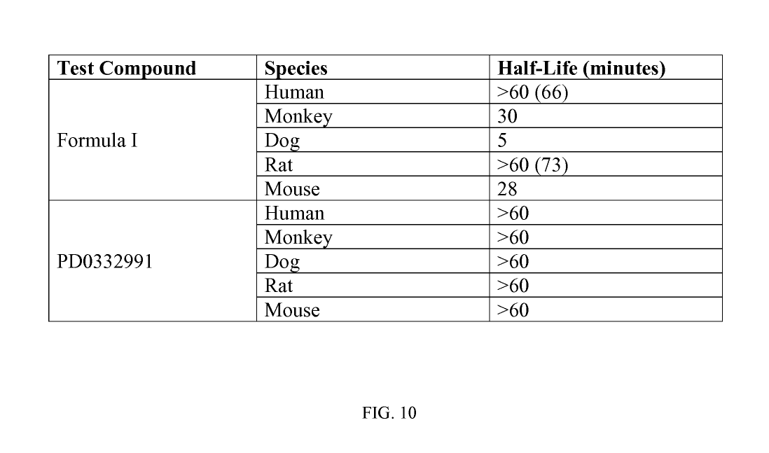

Further experiments were completed with Formula I and PD0332991 to compare the metabolic stability (half-life) in human, dog, rat, monkey, and mouse liver microsomes. As shown in FIG. 10, when analyzing the stability of the compounds in liver microsomes across species, the determinable half-life of Formula I is shorter in each species compared to that reported for PD0332991. Furthermore, as previously described above and in FIG. 8, it appears that PD0332991 also has an extended enzymatic half-life, as evidenced by the production of a pronounced cell cycle arrest in human cells lasting more than forty hours even after compound is removed from the cell culture media (i.e., in an in vitro wash-out experiment). As further shown in FIG. 8, removal of the compounds described herein from the culture media leads to a rapid resumption of proliferation, consistent with a rapid enzymatic off rate. These differences in enzymatic off rates translate into a marked difference in pharmacodynamic (PD) effect, as shown in FIGS. 5, 6C, and 7. As shown, a single oral dose of PD0332991 produces a 36+ hour growth arrest of hematopoietic stem and progenitor cells (HSPCs) in murine bone marrow, which is greater than would be explained by the 6 hour PK half-life of PD0332991 in mice. In contrast, the effect of Formula I is much shorter, allowing a rapid re-entry into the cell cycle, providing exquisite in vivo control of HSPC proliferation.

### Example 13

### Formula I Inhibits Proliferation of Hematopoietic Stem and/or Progenitor Cells (HSPCs)

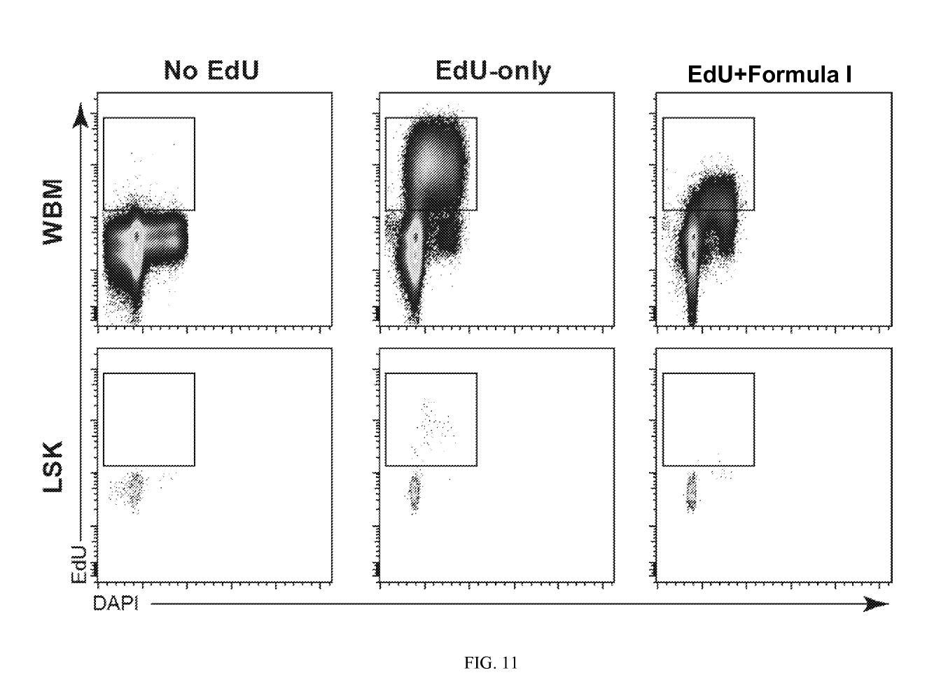

To characterize the effects of Formula I treatment on proliferation of the different mouse hematopoietic cells, 8-week-old female C57B1/6 mice were given a single dose of vehicle alone (20% Solutol) or Formula I (150 mg/kg) by oral gavage. Ten-hours later, all mice were given a single i.p. injection of 100 mcg EdU (5-ethynyl-2′-deoxyuridine) to label cells in S-phase of the cell cycle. All treated mice were euthanized 2 hours after EdU injection, bone marrow cells were harvested and processed for flow cytometric analysis of EdU-incorporation (FIG. 11).

In FIG. 11, representative contour plots show proliferation in WBM (whole bone marrow; top) and HSPCs (hematopoietic stem and progenitor cells; LSK; bottom), as measured by EdU incorporation for cells with no treatment, EdU treatment only, or EdU plus Formula I treatment. Formula I was found to reduce proliferation of whole bone marrow and hematopoietic stem and progenitor cells.

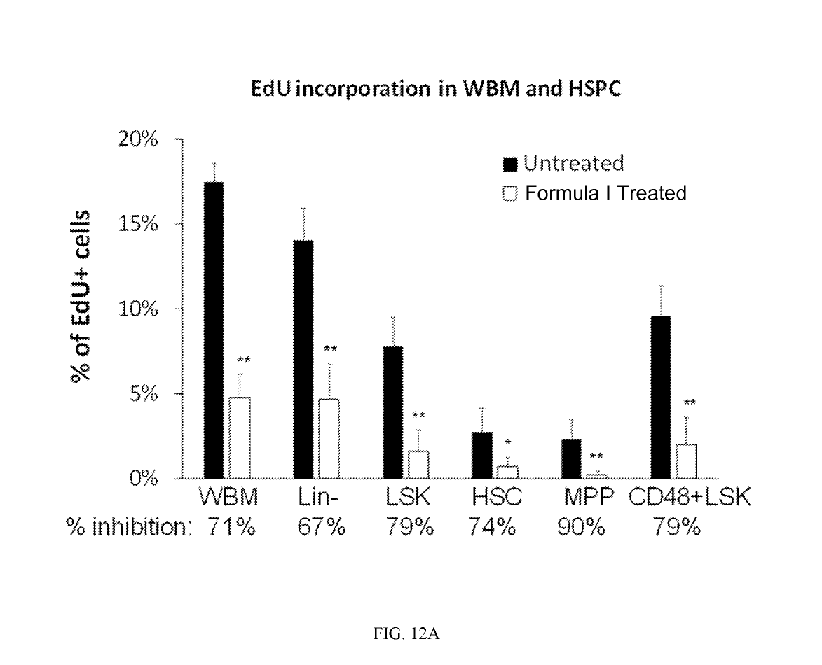

Compared to vehicle-treated mice, Formula I treated mice showed significantly less EdU-positive (EdU⁺) cells in all hematopoietic lineages analyzed. The reduction in EdU⁺ cell frequency is most likely due to reduced S-phase entry, which is consistent with the fact that Formula I potently inhibits CDK4/6 activity. Overall, Formula I treatment caused ˜70% reduction of EdU⁺ cell frequency in unfractionated whole bone marrow cells (See FIG. 11 and FIG. 12). In the hematopoietic stem and progenitor cells (HSPC), Formula I treatment resulted in potent cell cycle arrest of hematopoietic stem cells (HSC, 74% inhibition), the most primitive cells in the entire hematopoietic lineage hierarchy, as well as multipotent progenitors (MPP, 90% inhibition), the immediate downstream progeny of HSCs (FIG. 12A).

As shown in FIG. 12B, further down the lineage differentiation hierarchy, proliferation of the lineage restricted myeloid (CMP, GMP and MEP) and lymphoid progenitors (CLP) were also significantly inhibited by Formula I, showing between a 76-92% reduction in EdU⁺ cell frequency.

### Example 14

### Formula I Inhibits Proliferation of Differentiated Hematopoietic Cells

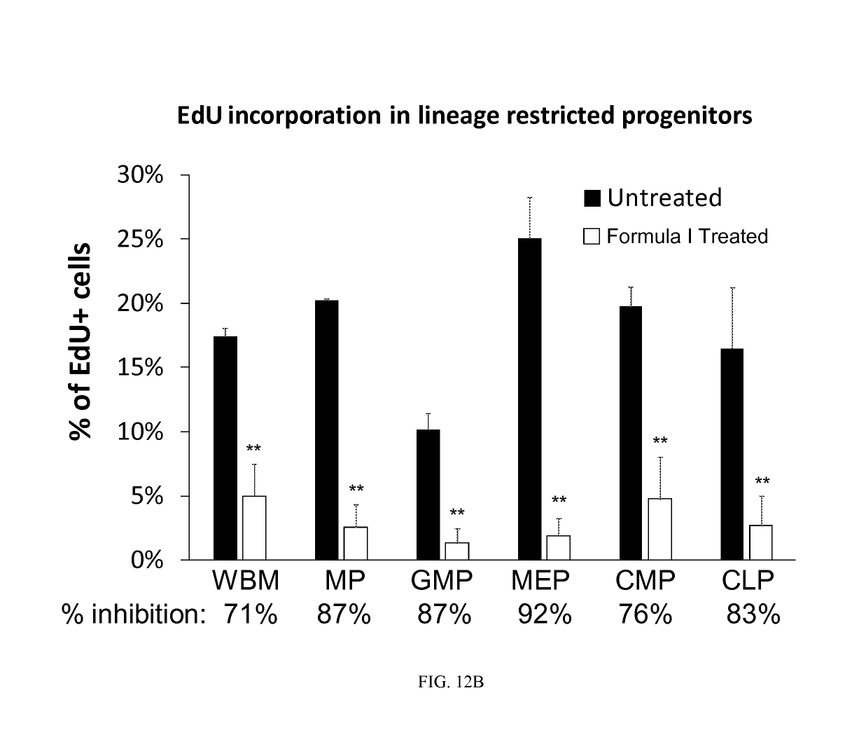

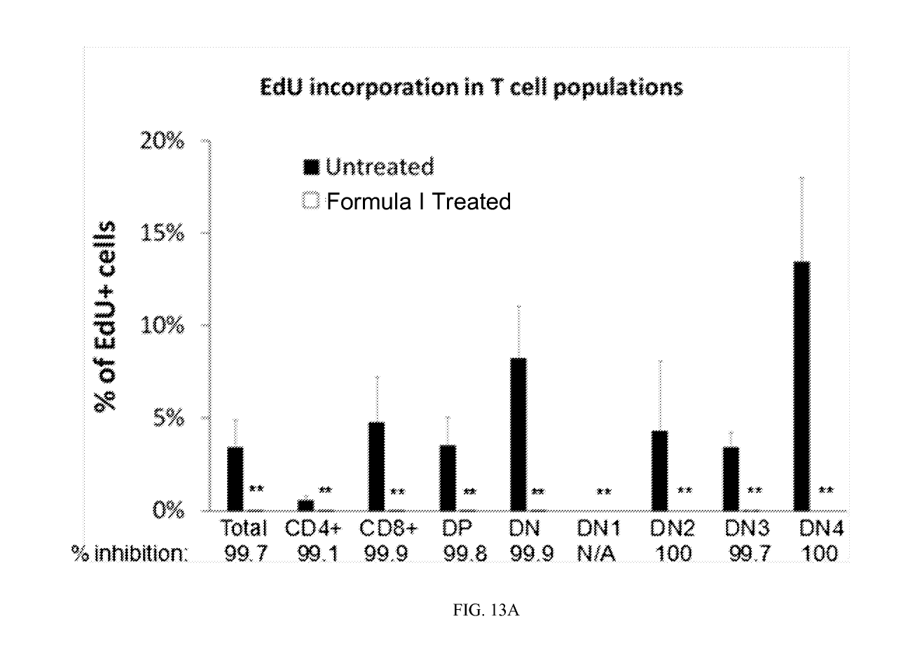

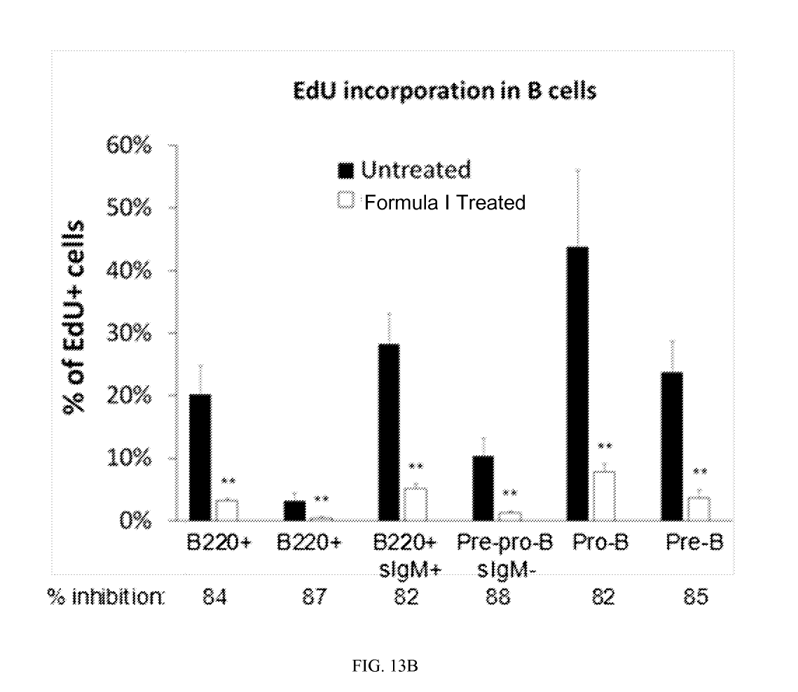

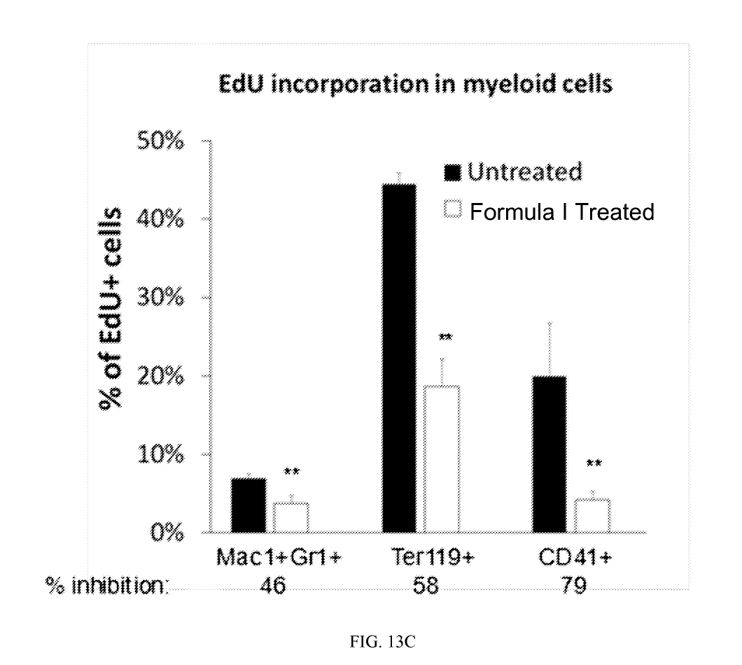

Using the same experimental protocol as discussed in Example 13 above and shown in FIGS. 11 and 12, the effects of Formula I on the proliferation of differentiated hematopoietic cells was investigated. The resulting effect of Formula I in differentiated hematopoietic cells was more variable than that seen in HSPCs. While T and B cell progenitors are highly sensitive to Formula I (>99% and >80% reduction in EdU⁺ cell frequencies respectively), proliferation of differentiated myeloerythroid cells are more resistant to Formula I, with Mac1⁺G1⁺ myeloid cells showing 46% reduction in EdU⁺ cell frequency, and Ter119⁺ erythroid cells showing 58% reduction in EdU⁺ cell frequency (FIG. 13). Together, these data suggest that while all hematopoietic cells are sensitive to Formula I-induced cell cycle arrest, the degree of inhibition varies among different cell lineages, with myeloid cells showing a smaller effect of Formula I on cell proliferation than seen in the other cell lineages.

### Example 15

### Radiomitigation Effects of CDK4/6 Inhibitors

The principal acute toxicities of total body irradiation (TBI) at doses less than 10 Gy are hematologic manifestions such as granulocytopenia, anemia, thrombocytopenia and lymphopenia. At higher doses of IR exposure, intestinal, cutaneous and neurologic toxicities additionally become significant contributors to morbidity and mortality, but the hematologic syndrome has been the principal complication faced by immediate survivors of a mass casualty radiologic disaster. Due to the important role that CDK4/6 plays in regulating the cell cycle at the G1 to S phase transition, CDK4/6 inhibitors were tested for their ability to protect cells from DNA damage and apoptosis induced by irradiation.

DNA damage was determined using the g-H2A.X assay and apoptosis was determined with a Caspase 3/7 assay. For the g-H2AX assay, tHS68 cells were fixed and stained using the g-H2A.X Phosphorylation Assay Kit (Flow Cytometry; Millipore, Temecula, Calif.) by the manufacturer's instructions. g-H2AX-positive tHDF cells were then quantified using a CyAn ADP Analyzer (Beckman Coulter, Indianapolis, Ind.) and FlowJo analysis software (Version 7.2.2; Tree Star, Ashland, Oreg.). For the in vitro caspase 3/7 assay, tHDF cells were analyzed directly in the 96-well plates 24 hours after radiation or staurosporine treatment. Caspase 3/7 activation was measured using the Caspase-Glo 3/7 Assay System (Promega, Madison, Wis.) by following the manufacturer's instructions.

For the g-H2AX assay, 30,000 cells were plated per well in 12-well plates. For the caspase 3/7 assay, 1,000 cells were plated per well in 96-well white wall clear bottom plates. Cells were incubated at 37° C. in a humidified atmosphere of 5% CO2 for 24 hours and then irradiated at 6 Gy, 8 Gy, or 10 Gy. Cells were then incubated at 37° C. in a humidified atmosphere of 5% CO2 with 100, 300, or 1,000 nM Formula I or dimethyl sulfoxide (Sigma-Aldrich) vehicle control for an additional 16 hours prior to analysis.

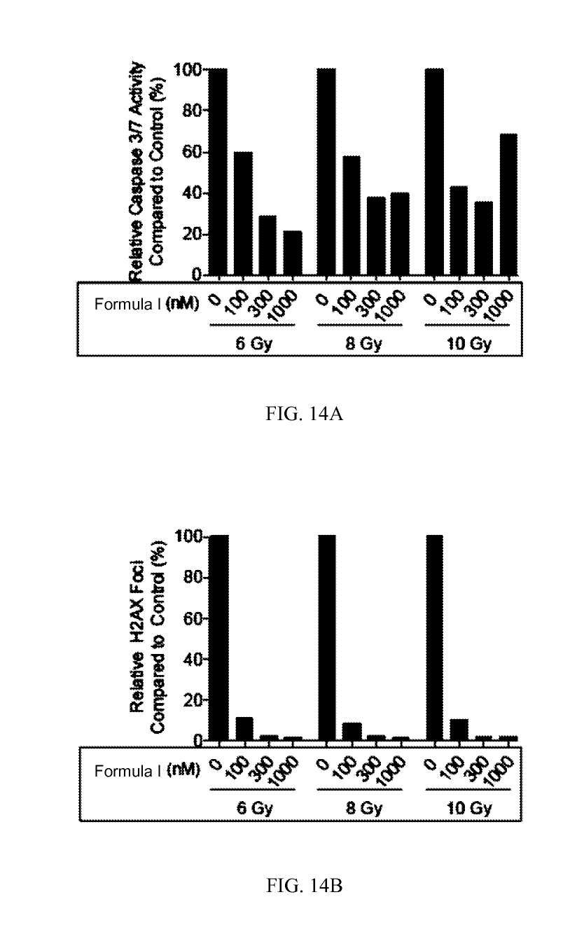

As shown in FIG. 14A, in vitro analysis of Formula I has demonstrated that it provides a dose dependent decrease in radiation induced apoptosis. As shown in FIG. 14B, in vitro analysis of Formula I has demonstrated that it provides a dose dependent decrease in radiation induced DNA damage.

### Example 16

### Radiomitigation Effects of CDK4/6 Inhibitors in a Mouse Model

Based on the radiomitigation effect seen in the in vitro experiments, Formula I was tested for mitigation of radiation-induced death in vivo in a mouse model. Wild-type mice, young adult (8-12 weeks of age) C57BL/6 (The Jackson Laboratory) or C3H (Harlan Sprague-Dawley) animals were used. Animals were irradiated using a 137Cs AECL GammaCell 40 Irradiator (Atomic Energy of Canada) or a XRAD320 (Precision XRay Inc.) biological irradiator. Experiments were carried out using the 137Cs source, unless otherwise noted. Mice were dosed at 150 mg/kg of Formula I by oral gavage 12 hours post irradiation for single dose studies. Mice were dosed at 150 mg/kg of Formula I by oral gavage 12 hours post irradiation and 24 hours post irradiation for two dose studies. Kaplan-Meier analysis of survival over the next 30 days for both treated and control groups were determined.

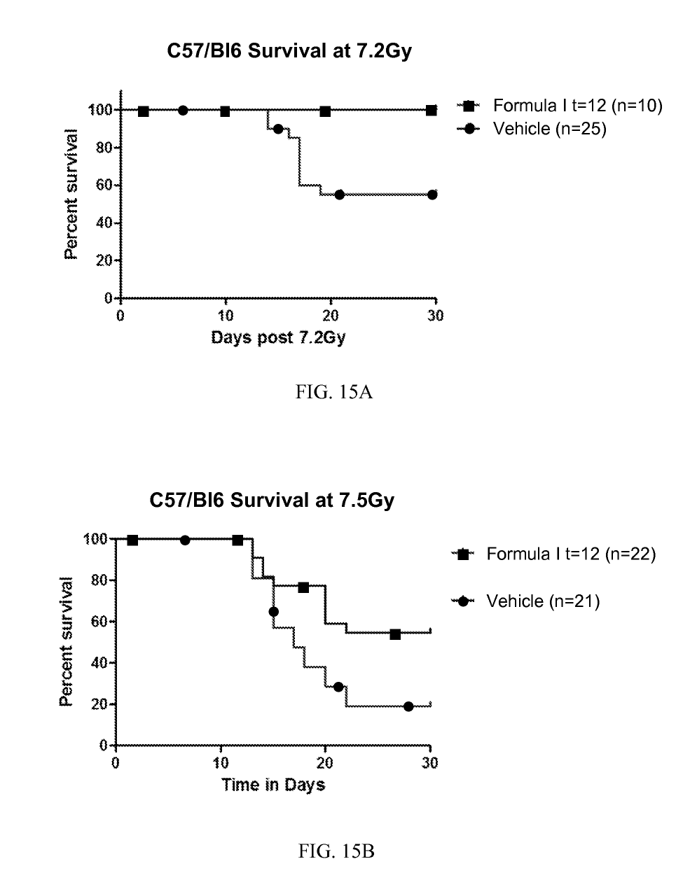

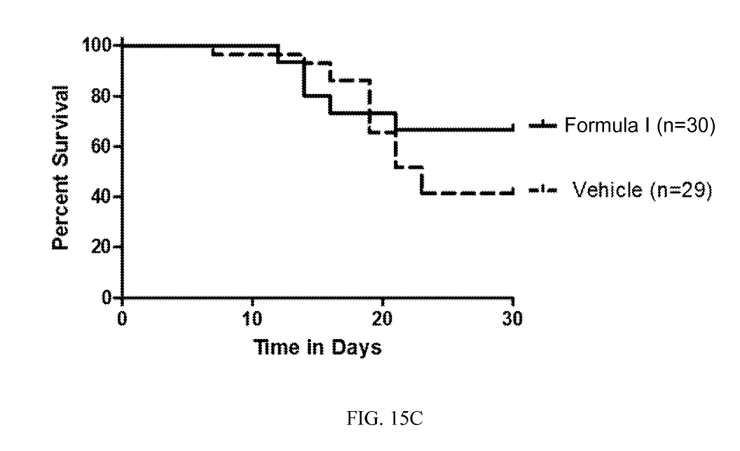

As shown in FIG. 15A, a single oral dose of Formula I (150 mg/kg) provided radiomitigation when administered 12 hours after exposure to 7.2 Gy. Additionally, a single oral dose of Formula I (150 mg/kg) provided a significant survival effect when administered 12 hours after exposure of 7.5 Gy (FIG. 15B). Survival was also enhanced when a second, equivalent dose of the drug was administered at 24 hours (FIG. 15C). These data further demonstrate the in vivo efficacy of Formula I to decrease the toxicity in bone marrow from DNA damaging insults.

### Example 17

### Preparation of Drug Product

The active compounds of the present invention can be prepared for intravenous administration using the following procedure. The excipients hydroxypropyl-beta-cyclodextrin and dextrose can be added to 90% of the batch volume of USP Sterile Water for Injection or Irrigation with stirring; stir until dissolved. The active compound in the hydrochloride salt form is added and stirred until it is dissolved. The pH is adjusted with 1N NaOH to pH 4.3+0.1 and 1N HCl can be used to back titrate if necessary. USP Sterile Water for Injection or Irrigation can be used to bring the solution to the final batch weight. The pH is next re-checked to ensure that the pH is pH 4.3+0.1. If the pH is outside of the range add 1N HCl or 1N NaOH as appropriate to bring the pH to 4.3+0.1. The solution is next sterile filtered to fill 50 or 100 mL flint glass vials, stopper, and crimped.

This specification has been described with reference to embodiments of the invention. The invention has been described with reference to assorted embodiments, which are illustrated by the accompanying Examples. The invention can, however, be embodied in different forms and should not be construed as limited to the embodiments set forth herein. Given the teaching herein, one of ordinary skill in the art will be able to modify the invention for a desired purpose and such variations are considered within the scope of the invention.

1. A method for reducing the effect of ionizing radiation exposure on hematopoietic stem cells and/or progenitor cells (HSPCs) in a subject undergoing at least three ionizing radiation treatments within a week for a cyclin dependent kinase 4/6 replication independent cancer, the method comprising:
a. administering to the subject an effective amount of a compound having the formula: 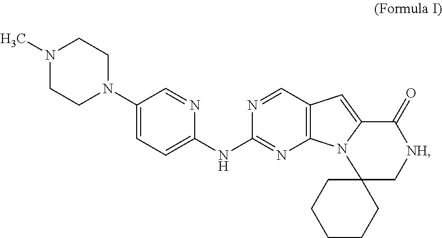 [SMILES: CN1CCN(c2ccc(Nc3ncc4cc5n(c4n3)C3(CCCCC3)CNC5=O)nc2)CC1]
or its pharmaceutically acceptable salt thereof; and,
b. exposing the subject to the ionizing radiation;
wherein the subject is administered an effective amount of the compound prior to exposing the subject to each of the at least three ionizing radiation treatments.

2. The method of claim 1, wherein the subject is a human.

3. The method of claim 1, wherein the subject is being treated for a CDK4/6 replication independent cancer selected from small cell lung cancer, triple-negative breast cancer, HPV-positive head and neck cancer, retinoblastoma, Rb-negative bladder cancer, Rb negative prostate cancer, osteosarcoma or cervical cancer.

4. The method of claim 3, wherein the cancer is small cell lung cancer.

5. The method of claim 3, wherein the cancer is triple-negative breast cancer.

6. The method of claim 3, wherein the cancer is HPV-positive head and neck cancer, retinoblastoma.

7. The method of claim 3, wherein the cancer is Rb-negative bladder cancer.

8. The method of claim 3, wherein the cancer is Rb negative prostate cancer.

9. The method of claim 3, wherein the cancer is osteosarcoma.

10. The method of claim 3, wherein the cancer is cervical cancer.

11. The method of claim 1, wherein the compound is administered to the subject less than about 24 hours prior to exposure to the ionizing radiation.

12. The method of claim 1, wherein the compound is administered to the subject prior to exposure to the ionizing radiation such that the compound reaches peak serum levels during exposure to the ionizing radiation.

13. The method of claim 1, wherein the compound is administered to the subject less than about 4 hours prior to exposure to the ionizing radiation.

14. The method of claim 1, further comprising administering to the subject a hematopoietic growth factor.

15. The method of claim 14, wherein the hematopoietic growth factor is granulocyte colony stimulating factor (G-CSF), granulocyte-macrophage colony stimulating factor (GM-CSF), M-CSF (macrophage colony stimulating factor), thrombopoietin, interleukin (IL)-12, SCF (stem cell factor) and erythropoietin (EPO).

16. The method of claim 15, wherein the hematopoietic growth factor is G-CSF.

17. The method of claim 15, wherein the hematopoietic growth factor is GM-CSF.

18. The method of claim 15, wherein the hematopoietic growth factor is EPO.

19. The method of claim 15, wherein the hematopoietic growth factor is thrombopoietin.

20. The method of claim 1, wherein subject is exposed to the ionizing radiation 3 times within a week.

21. The method of claim 1, wherein the subject is exposed to the ionizing radiation 4 times within a week.

22. The method of claim 1, wherein the subject is exposed to the ionizing radiation 5 times a week.
